(19) United States
(12) Patent Application Publication
(10) Pub. No.: US 2009/0280109 A1
(43) Pub. Date: Nov. 12, 2009

(54) ILT3 and ILT4-related compositions and methods

(75) Inventors: Nicole Suciu-Foca, New York, NY (US); Zhuoru Liu, New York, NY (US); Chih-Chao Chang, Scarsdale, NY (US); Raffaello Cortesini, New York, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: The Trustees of Columbia University in the City of New York, New York, NY (US)

(21) Appl. No.: 12/072,119

(22) Filed: Feb. 22, 2008

Related U.S. Application Data
(63) Continuation of application No. 10/479,381, filed on Jun. 24, 2004, now abandoned.
(60) Provisional application No. 60/300,731, filed on Jun. 25, 2001.

(51) Int. Cl. A61K 39/395

(52) U.S. Cl. 424/130.1

(57) ABSTRACT

This invention provides compositions which comprise at least two of a CD4+CD25+ cell, IL-10, a CD8+CD28− cell and a vitamin D₃ analog. This invention also provides methods for generating a tolerogenic antigen-presenting cell, and increasing the expression of ILT3 and/or ILT4 by an antigen-presenting cell. This invention further provides methods for inhibiting the onset of or treating the rejection of an antigenic substance and inhibiting the onset of or treating an autoimmune disease in a subject. This invention further provides methods for treating and preventing AIDS, cancer, and Hepatitis C-related disorders, and for identifying agents useful for such purposes. Finally, this invention provides related compositions and kits.

This application claims priority of U.S. Ser. No. 60/300,731, filed Jun. 25, 2001, and of U.S. Ser. No. 10/056,922, filed Jan. 24, 2002, the contents of which are hereby incorporated by reference into the present application.

This invention was made with support under Grant No. AI25210-15 from the National Institutes of Health. Accordingly, the United States Government has certain rights in the invention.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

The inhibitory activity shown by regulatory T (T_{R}) lymphocytes is believed to be central to the prevention of autoimmune diseases, allergies, transplant rejection and immune-deficiency disorders. Recent evidence indicates that multiple types of T_{R} cells may exist. Different subsets of CD4⁺ and CD8⁺ T lymphocytes show regulatory activities that are mediated by immunosuppressive cytokines or by contact-dependent mechanisms (1-4). In both humans and rodents one of the best-characterized populations of T_{R} cells are the CD4⁺ CD25⁺ lymphocytes. After T cell receptor (TCR)-triggering, CD4⁺ CD25⁺ T_{R} cells inhibit immune responses in vivo and in vitro via an antigen-presenting cell (APC)-independent mechanism. This occurs in an antigen-nonspecific and major histocompatibility complex (MHC)-nonrestricted manner (1-6). Human as well as murine CD4⁺CD25⁺ T_{R} cells are anergic and express intracellular cytotoxic T lymphocyte-associated antigen 4 (CTLA-4), a costimulatory receptor which delivers a negative or “off” signal to T cells (6, 7). CTLA-4 may account for the ability of CD4⁺CD25⁺ T cells to suppress immune responses in vivo (8). There is evidence that CD4⁺CD25⁺ T cell-mediated suppression of conventional CD4⁺CD25⁻ T cell activation in response to alloantigen, immobilized anti-CD3 and phytohemagglutinin (PHA) stimulation is based on contact-dependent, cytokine-independent, T cell-to-T cell interaction (5, 9). One hypothesis suggests that after TCR-mediated activation, CD4⁺CD25⁺ T cells express cell surface molecule(s) that mediate suppression by binding to a counter-receptor on CD4⁺CD25⁻ T cells. This counter-receptor may also require induction by TCR ligation (3).

A distinct subset of CD4⁺ T_{R} cells, isolated by expanding human T cells primed with alloantigens in the presence of interleukin 10 (IL-10) was termed type 1 T_{R} (T_{R}1) cells (10). These cells inhibit both naïve and memory T cells in an antigen-specific manner via a mechanism that is partially dependent on the production of the immunoregulatory cytokines IL-10 and transforming growth factor-β (TGF-β) (10). Similarly, within the human CD8⁺ subset, there exist antigen-specific T_{R} cells that suppress CD4⁺ T helper (T_{H}) cell reactivity by producing IL-10 (11).

It has been previously shown that there is a distinct population of human T_{R} cells which are characterized by their CD8⁺CD28⁻ phenotype (12-17) and are referred to as T suppressor (T_{S}) cells (12-18). Like the CD4⁺ T_{R} cells, CD8⁺CD28⁻ T_{S} cells can be generated in vitro after multiple rounds of stimulation of human peripheral blood mononuclear cells (PBMCs) with either allogeneic-(12) or xenogeneic-donor APCs(13). Similarly, CD8⁺CD28⁻ T_{S} can be generated in vitro by priming PBMCs with self-APCs pulsed with nominal antigens such as MHC antigens or tetanus toxin (14). CD8⁺CD28⁻ T_{S} cells are MHC class I-restricted and suppress antigen-specific CD4⁺ T_{H} cell responses, inhibiting their capacity to produce IL-2 and preventing up-regulation of CD40 ligand (CD40L)(12-15). Inhibition of CD4⁺ T_{H} cell proliferation is not caused by killing either APCs or CD4⁺ T_{H} cells. Neither is the suppressor effect mediated by the production of cytokines; instead it requires direct interactions between CD8⁺CD28⁻ T_{S} cells and the APCs used for priming (12, 13). In this system, the APCs act as a bridge between CD8⁺CD28⁻ T_{S} cells—which recognize peptide-MHC class I complexes on their cell surfaces—and CD4⁺ T_{H} cells—which recognize peptide-MHC class II complexes on their cell surfaces(12). CD8⁺CD28⁻ T_{S} cells inhibit CD40-mediated up-regulation of costimulatory molecules such as CD80 and CD86 on APCs that present the peptide-MHC class I complexes to which the CD8⁺CD28⁻ T_{S} cells have been previously primed (12, 13, 16). The suppressed APCs are rendered unable to induce and sustain the full program of CD4⁺ T_{H} cell activation due, at least in part, to the inhibition of NF-κB activation and transcription of costimulatory molecules in APCs (17).

### SUMMARY OF THE INVENTION

This invention provides a first composition which comprises at least two of a CD4+CD25+ cell, IL-10, a CD8+CD28− cell, and/or a vitamin D3 analog, in prophylactically or therapeutic amounts.

This invention further provides a composition which comprises the first instant composition and a pharmaceutically acceptable carrier.

This invention further provides method for generating a tolerogenic antigen-presenting cell which comprises contacting the cell with an effective amount of IL-10, a CD4+CD25+ and/or a vitamin D3 analog.

This invention further provides a method for increasing the expression of ILT3 and/or ILT4 by an antigen-presenting cell which comprises contacting the cell with an effective amount of IL-10, a CD4+CD25+ cell and/or a vitamin D3 analog.

This invention further provides a method for inhibiting the onset of rejection of an antigenic substance in a subject, which comprises administering to the subject a prophylactically effective amount of IL-10, a CD4+CD25+ cell, and/or a vitamin D3 analog.

This invention further provides a method for treating the rejection of an antigenic substance in a subject, which comprises administering to the subject a therapeutically effective amount of IL-10, a CD4+CD25+ cell, and/or a vitamin D3 analog.

This invention further provides a method for inhibiting the onset of an autoimmune disease in a subject, which comprises administering to the subject a prophylactically effective amount of IL-10, a CD4+CD25+ cell, and/or a vitamin D3 analog.

This invention further provides a method for treating autoimmune disease in a subject, which comprises administering to the subject a therapeutically effective amount of IL-10, CD4+CD25+ cell, and/or vitamin D3 analog.

This invention further provides a second composition of matter comprising an agent that specifically binds to ILT3 and/or ILT4.

This invention further provides a composition which comprises the second instant composition and a pharmaceutically acceptable carrier.

This invention further provides a method for decreasing the expression of ILT3 and/or ILT4 by an antigen-presenting cell which comprises contacting the cell with the second instant composition.

This invention further provides a method for inhibiting the onset of AIDS or cancer in a subject, which comprises administering to the subject a prophylactically effective amount of the second instant composition and a pharmaceutically acceptable carrier.

This invention further provides a method for treating AIDS or cancer in an afflicted subject, which comprises administering to the subject a therapeutically effective amount of the second instant composition and a pharmaceutically acceptable carrier.

This invention further provides a method for inhibiting the onset of a Hepatitis C-related disorder in a subject infected with the Hepatitis C virus, which comprises administering to the subject a prophylactically effective amount of the second instant composition and a pharmaceutically acceptable carrier.

This invention further provides a method for treating a Hepatitis C-related disorder in a subject infected with the Hepatitis C virus, which comprises administering to the subject a prophylactically effective amount of the second instant composition and a pharmaceutically acceptable carrier.

This invention further provides a method for determining the degree to which a subject is immunocompromised, which comprises determining the expression level of ILT3 and/or ILT4 in antigen-presenting cells of the subject and comparing the expression level so determined to the ILT3 and/or ILT4 expression level in antigen-presenting cells of a subject whose immune system is normal or compromised to a known degree.

This invention further provides a method for determining the likelihood that a subject's immune system will reject an antigenic substance if introduced into the subject, which comprises determining the expression level of ILT3 and/or ILT4 in the antigen-presenting cells of the subject, and comparing the expression level so determined to the expression level of ILT3 and/or ILT4 determined in antigen-presenting cells of a subject whose immune system has a known likelihood for rejecting the antigenic substance.

This invention further provides a method for determining whether an agent is an immunosuppressant or an immunostimulant which comprises (a) contacting the agent with an antigen-presenting cell and (b) determining the resulting expression level of ILT3 and/or ILT4 in the cell, an increase of ILT3 and/or ILT4 expression resulting from step (a) indicating that the agent is an immunosuppressant, and a decrease of ILT3 and/or ILT4 expression resulting from step (a) indicating that the agent is an immunostimulant.

This invention further provides a method for determining whether an agent is an immunosuppressant or an immunostimulant which comprises (a) administering the agent to a subject and (b) determining the resulting expression level of ILT3 and/or ILT4 in the subject's antigen-presenting cells, an increase of ILT3 and/or ILT4 expression resulting from step (a) indicating that the agent is an immunosuppressant, and a decrease of ILT3 and/or ILT4 expression resulting from step (a) indicating that the agent is an immunostimulant.

Finally, this invention also provides for a kit practicing any of the above-identified methods, comprising (a) an agent useful for quantitating ILT3 and/or ILT4 or nucleic acid encoding same, and (b) instructions for use.

### BRIEF DESCRIPTION OF THE FIGURES

FIGS. 1A and B

CD8+CD28− T_{S} inhibit CD4+ T_{H} proliferation and render APC tolerogenic. (a) The addition of monoclonal anti-ILT3 or a cocktail of monoclonal anti-ILT4 plus anti-HLA class I to cultures containing CD4+ T_{H}, CD8+CD28− T_{S} and stimulating APC partially abrogates the T_{S} effect on T_{H} proliferation; (b) rIL2 restores T_{H} proliferation in response to APC tolerized by exposure to T_{S}.

FIGS. 2A-C

CD8+CD28− T_{S} upregulate the expression of both ILT3 and ILT4 on APC. (a). ILT3 and ILT4 mRNA are increased in APC co-cultured with CD8+CD28− T_{S} (b) Time course of ILT3 and ILT4 mRNA induction in APC co-cultured with CD8+CD28− T_{S}. (c) Expression of ILT3, ILT4 and CD86 on CD14+ monocytes and CD11c⁺ HLA DR⁺ DC before and after exposure to CD8+CD28− T_{S}.

FIGS. 3A-D

ILT3 and ILT4 transduction of KG1 APC. (a) Map of MIG retroviral expression vectors encoding ILT3 and ILT4. (b) Fluorescence histogram of ILT3 and ILT4 expression on the surface of ILT3-MIG-KG1, ILT4-MIG-KG1 or MIG-KG1 control. (c) CD80 expression on the cell surface of MIG-KG1, ILT3-MIG-KG-1 and ILT4-MIG-KG1 in cultures with or without KG1-primed CD4+ T_{H}. (d) Proliferative responses of naïve and memory CD4+ T_{H} to ILT3-MIG-KG1 and ILT4-MIG-KG1 in cultures with or without anti-ILT3, or rIL2.

FIGS. 4A and 4B

Molecular and functional changes accompany ILT3 expression in KG1 APCs. (a) NF-kB activation induced by CD4+ T_{H} in KG1 APC is suppressed by CD8+CD28− T_{S} as determined by EMSA using Sp1 specific probe as control. 1 mg of nuclear extract from KG1 APC was used. (b) Inhibition of NF-kB activation in ILT3-MIG-KG1 clone A and clone B.

FIGS. 5A-C

Expression of ILT3 and ILT4 in APC from the spleen of transplant donors after preincubation with recipient's CD8+CD28− T cells. (a) ILT3 and ILT4 mRNA in CD14+ donor splenocytes treated and untreated with CD8+CD28− T_{S} cells from the corresponding heart transplant recipient. (b) ILT3 and ILT4 expression on the cell surface of CD14+ splenocytes from the same donors and from HLA mismatched controls, before and after incubation with recipients' CD8+CD28− T cells. (c) CD86 expression on donor CD14+ splenocytes incubated with CD40-L (D1.1) transfected cells in the presence or absence of CD8+CD28− T cells from the corresponding recipient.

FIG. 6

Cytotoxic activity of CD8+ T cells from recipient with acute rejection. Annexin V and PI staining of CD20+ and CD14+ splenocytes from the transplant donor incubated with or without CD8+CD28− T cells or unfractionated CD8+ T cells from the corresponding recipient.

FIGS. 7A-C

The relationship between CD8+CD28− T cells and ILT3/ILT4 expression on monocytes from immunologically deficient, HIV-infected patients. (a) Phenotypic characterization of peripheral blood T lymphocytes in HIV-infected vs. Non-infected. (b) Percentage of monocytes expressing ILT4. (c) ILTa mRNA levels measured by semiquantitative RT-PCR in CD14+ cells of HIV-infected and healthy individuals.

FIG. 8

Direct correlation between the frequency of CD8⁺CD28⁻ T cells and the percentage of ILT4⁺ monocytes in the population of HIV infected patients.

### DETAILED DESCRIPTION OF THE INVENTION

This invention provides a first composition which comprises at least two of a CD4+CD25+ cell, IL-10, a CD8+CD28− cell and/or a vitamin D₃ analog, in prophylactically or therapeutic amounts. In the preferred embodiment, the CD4+CD25+ cell is a CD4+CD25+Ro+ cell, and the CD8+CD28− cell is a CD8+CD28−CD27+ cell. In one embodiment, the composition further comprises a pharmaceutically acceptable carrier. In another embodiment, the CD8+CD28− and CD4+CD25+ cells and IL-10 are human.

This invention further provides a method for generating a tolerogenic antigen-presenting cell which comprises contacting the cell with an effective amount of IL-10, a CD4+CD25+ cell and/or a vitamin D₃ analog. In an embodiment, the antigen-presenting cell is a human antigen-presenting cell. The contacting can be performed, for example, in vivo, ex vivo, or in vitro. In another embodiment, the method further comprises contacting the antigen-presenting cell with a CD8+CD28− cell. In another embodiment, the antigen-presenting cell is a dendritic cell or a monocyte.

This invention further provides a method for increasing the expression of ILT3 and/or ILT4 by an antigen-presenting cell which comprises contacting the cell with an effective amount of IL-10, a CD4+CD25+ cell and/or a vitamin D₃ analog. In one embodiment, the antigen-presenting cell is a human antigen-presenting cell. In another embodiment, the contacting is performed in vivo, ex vivo, or in vitro. In a further embodiment, the method further comprises contacting the antigen-presenting cell with a CD8+CD28− cell. In a further embodiment, the antigen-presenting cell is a dendritic cell or a monocyte.

This invention further provides a method for inhibiting the onset of rejection of an antigenic substance in a subject, which comprises administering to the subject a prophylactically effective amount of IL-10, a CD4+CD25+ cell, and/or a vitamin D₃ analog. In one embodiment, the antigenic substance is a transplanted cell, tissue or organ. In another embodiment, the antigenic substance is xenogeneic, allogeneic, and/or a prosthetic device. In the preferred embodiment, the subject is human.

This invention further provides a method for treating the rejection of an antigenic substance in a subject, which comprises administering to the subject a therapeutically effective amount of IL-10, a CD4+CD25+ cell, and/or a vitamin D₃ analog. In one embodiment, the antigenic substance is a transplanted cell, tissue or organ. In another embodiment, the antigenic substance is xenogeneic, allogeneic, and/or a prosthetic device. In the preferred embodiment, the subject is human.

This invention further provides a method for inhibiting the onset of an autoimmune disease in a subject, which comprises administering to the subject a prophylactically effective amount of IL-10, a CD4+CD25+ cell, and/or a vitamin D₃ analog. In one embodiment, the disease is selected from the group consisting of autoimmune encephalomyelitis, lupus, rheumatoid arthritis, multiple sclerosis, myasthenia gravis, psoriasis and Crohn's disease. In the preferred embodiment, the subject is human.

This invention further provides a method for treating autoimmune disease in a subject, which comprises administering to the subject a therapeutically effective amount of IL-10, CD4+CD25+ cell, and/or vitamin D3 analog. In one embodiment, the disease is selected from the group consisting of autoimmune encephalomyelitis, lupus, rheumatoid arthritis, multiple sclerosis, myasthenia gravis, psoriasis and Crohn's disease. In another embodiment, the subject is human.

This invention further provides a second composition of matter comprising an agent that specifically binds to ILT3 and/or ILT4. In one embodiment, the agent is an anti-ILT3 or ILT4 antibody, or antigen-binding portion thereof.

This invention further provides a composition which comprises the second composition and a pharmaceutically acceptable carrier.

This invention further provides a method for decreasing the expression of ILT3 and/or ILT4 by an antigen-presenting cell which comprises contacting the cell with the second composition. In one embodiment, the antigen-presenting cell is a human antigen-presenting cell. In another embodiment, the contacting is performed in vivo, ex vivo, or in vitro. In another embodiment, the antigen-presenting cell is a dendritic cell or a monocyte.

This invention further provides a method for inhibiting the onset of AIDS or cancer in a subject, which comprises administering to the subject a prophylactically effective amount of the second composition. In the preferred embodiment, the subject is human.

This invention further provides a method for treating AIDS or cancer in an afflicted subject, which comprises administering to the subject a therapeutically effective amount of the second composition. In the preferred embodiment, the subject is human.

This invention further provides a method for inhibiting the onset of a Hepatitis C-related disorder in a subject infected with the Hepatitis C virus, which comprises administering to the subject a prophylactically effective amount of the second composition. In the preferred embodiment, the subject is human. Hepatitis C-related disorders include by example cirrhosis and liver cancer.

This invention further provides a method for treating a Hepatitis C-related disorder in a subject infected with the Hepatitis C virus, which comprises administering to the subject a prophylactically effective amount of the second composition. In the preferred embodiment, the subject is human.

This invention further provides a method for determining the degree to which a subject is immunocompromised, which comprises determining the expression level of ILT3 and/or ILT4 in antigen-presenting cells of the subject and comparing the expression level so determined to the ILT3 and/or ILT4 expression level in antigen-presenting cells of a subject whose immune system is normal or compromised to a known degree. In one embodiment, the antigen-presenting cell is a dendritic cell or a monocyte. In the preferred embodiment, the subject is human. In one embodiment, determining the expression level of ILT3 and/or ILT4 comprises determining the level of mRNA encoding same. In another embodiment, determining the expression level of ILT3 and/or ILT4 comprises determining the level of ILT3 and/or ILT4 protein. In the methods of this invention, determining the amount of ILT3 and ILT4 expression can be performed, for example, using whole blood, isolated APCs, or isolated monocytes.

This invention further provides a method for determining the likelihood that a subject's immune system will reject an antigenic substance if introduced into the subject, which comprises determining the expression level of ILT3 and/or ILT4 in the antigen-presenting cells of the subject, and comparing the expression level so determined to the expression level of ILT3 and/or ILT4 determined in antigen-presenting cells of a subject whose immune system has a known likelihood for rejecting the antigenic substance. In one embodiment, the antigen-presenting cell is a dendritic cell or a monocyte. In the preferred embodiment, the subject is human. In one embodiment, determining the expression level of ILT3 and/or ILT4 comprises determining the level of mRNA encoding same. In another embodiment, determining the expression level of ILT3 and/or ILT4 comprises determining the level of ILT3 and/or ILT4 protein. In an embodiment, the antigenic substance is a transplanted cell, tissue or organ. The antigenic substance can be, for example, xenogeneic, allogeneic, or a prosthetic device.

This invention further provides a method for determining whether an agent is an immunosuppressant or an immunostimulant which comprises (a) contacting the agent with an antigen-presenting cell and (b) determining the resulting expression level of ILT3 and/or ILT4 in the cell, an increase of ILT3 and/or ILT4 expression resulting from step (a) indicating that the agent is an immunosuppressant, and a decrease of ILT3 and/or ILT4 expression resulting from step (a) indicating that the agent is an immunostimulant. In the preferred embodiment, the antigen-presenting cell is human. In another embodiment, the antigen-presenting cell is a dendritic cell or a monocyte. In one embodiment, determining the expression level of ILT3 and/or ILT4 comprises determining the level of mRNA encoding same. In another embodiment, determining the expression level of ILT3 and/or ILT4 comprises determining the level of ILT3 and/or ILT4 protein.

This invention further provides a method for determining whether an agent is an immunosuppressant or an immunostimulant which comprises (a) administering the agent to a subject and (b) determining the resulting expression level of ILT3 and/or ILT4 in the subject's antigen-presenting cells, an increase of ILT3 and/or ILT4 expression resulting from step (a) indicating that the agent is an immunosuppressant, and a decrease of ILT3 and/or ILT4 expression resulting from step (a) indicating that the agent is an immunostimulant. In one embodiment, determining the expression level of ILT3 and/or ILT4 comprises determining the level of mRNA encoding same. In another embodiment, determining the expression level of ILT3 and/or ILT4 comprises determining the level of ILT3 and/or ILT4 protein.

Finally, this invention provides a kit for practicing the above-identified methods, comprising (a) an agent useful for quantitating ILT3 and/or ILT4 or nucleic acid encoding same, and (b) instructions for use. In one embodiment, the agent is an antibody specific for ILT3 and/or ILT4. In another embodiment, the agent is a nucleic acid that specifically hybridizes to a nucleic acid encoding ILT3 and/or ILT4.

This invention will be better understood from the Experimental Details which follow. However, one skilled in the art will readily appreciate that the specific methods and results discussed are merely illustrative of the invention as described more fully in the claims which follow thereafter.

The abbreviations used herein are: TCL—T cell line; Th—T helper cell; Ts—T suppressor cell; PBMC—peripheral blood mononuclear cell; APC—antigen-presenting cell; DCs—dendritic cells; APCs—antigen-presenting cells; CD40L=CD40 Ligand; Mean fluorescence intensity—MFI; TNF—tumor necrosis factor; PE—Phycoerythrin; PI—Propidium Iodide; ILT3—immunoglobulin (Ig)-like transcript 3; ILT4—immunoglobulin (Ig)-like transcript 4; KIR—killer cell inhibitory receptor; TCR—T cell receptor.

### EXPERIMENTAL DETAILS

### First Series of Experiments

### A. Experimental Synopsis

General

The immunoglobulin like transcripts ILT3 and ILT4 belong to a family of inhibitory receptors expressed by human monocytes and dendritic cells. We now demonstrate that CD8⁺CD28⁻ alloantigen specific T-suppressor cells induce the upregulation of ILT3 and ILT4 on monocytes and dendritic cells rendering these antigen presenting cells (APC) tolerogenic. Tolerogenic APC show reduced expression of costimulatory molecules and induce antigen specific unresponsiveness in CD4⁺ T helper cells. Study of human heart transplant recipients showed that rejection-free patients have circulating T-suppressor cells, which induce the upregulation of ILT3 and ILT4 in donor APC. These findings demonstrate an important mechanism of immune regulation.

Detailed

To gain a better insight into the precise molecular basis that underlies the anergizing capacity acquired by APCs exposed to CD8⁺CD28⁻ T_{S} cells, allospecific human T cell lines (TCLs) were generated and the CD8⁺CD28⁻ T_{S} cells from these lines were used to modulate the function of monocytes and immature dendritic cells (DCs). As an allostimulator, the myelomonocytic cell line KG1 were used; KG1 cells show many of the phenotypic characteristics of immature DCs (19). After exposure to CD8⁺CD28⁻ T_{S} cells, these APCs show increased expression of the genes encoding immunoglobulin (Ig)-like transcript 3 (ILT3) and ILT4. The inhibitory receptors ILT3 and ILT4, which are expressed by monocytes and DCs, belong to a family of Ig-like inhibitory receptors that are structurally and functionally related to killer cell inhibitory receptors (KIRs) (20-24). The subset of ILT receptors, which includes ILT3 and ILT4, displays a long cytoplasmic tail containing immunoreceptor tyrosine-based inhibitory motifs (ITIMs). These receptors mediate inhibition of cell activation by recruiting tyrosine phosphatase SHP-1 (20-24). Coligation of ILTs in monocytes inhibits Ca²⁺ mobilization and tyrosine phosphorylation triggered by antibody ligation of FcγRII (also known as CD32), HLA-DR and FcγRI (also known as CD64) (20). Although the ligand for ILT3 is unknown, ILT4 binds HLA-A, HLA-B, HLA-C and HLA-G (20, 22). The present study demonstrates that CD8⁺CD28⁻ T_{S} cells induce the up-regulation of ILT3 and ILT4 on monocytes and dendritic cells, rendering these APC capable of anergizing CD4⁺ T_{H} cells.

### B. Materials and Methods

Transplant Patients

Citrate-anticoagulated whole blood was from the recipients of cadaver donor heart transplants treated at New York Presbyterian Medical Center. The average number of HLA mismatches between the organ donors and transplant recipients was 2.6±1.4 for HLA-A and HLA-B and 1.8±0.2 for HLA-DR. All patients were treated with standard immunosuppressive therapy. Endomyocardial biopsies were done to monitor rejection in heart allograft recipients according to a standard time schedule as described (43).

Spleens from cadaver donors were obtained and used for histocompatibility testing; splenocyte suspensions were cryopreserved at the time of transplantation. All experiments were done in compliance with the relevant laws and institutional Good Clinical Practice guidelines and were Institutional Review Board-approved.

Generation of Alloreactive TCLs

PBMCs from healthy volunteers were separated from peripheral blood by Ficoll-Hypaque centrifugation. Responding PBMCs (1×10⁶/ml) were stimulated in 24-well plates with irradiated (1600 rad) APCs (0.5×10⁶/ml) obtained from allogeneic PBMCs via the depletion of CD2⁺ T cells. Alternatively, responding PBMCs were stimulated with irradiated (3000 rad) cells (0.5×10⁶/ml) from the KG1 myelomonocytic cell line. The KG1 cell line (ATCC, Manassas, Va.) expresses HLA-A30, HLA-B35, HLA-B51, HLA-BW4, HLA-BW6, HLA-DRB1*1101 and HLA-DRB1*1401.

Cells were cultured for 7 days in complete medium (RPMI 1640 supplemented with 10% human serum, 2 mM 1-glutamine and 50 μg/ml of gentamycin) (Gibco-BRL, Grand Island, N.Y.). After 7 days, responding cells were collected, washed and rechallenged with the original stimulating cells. Three days later, rIL-2 (Boehringer Mannheim, Indianapolis, Ind.) was added (10 U/ml) and the cultures were expanded for an additional 4 days. Proliferation assays were done on day 14.

Three-Day Proliferation Assay

Before testing, responding T cells were depleted of natural killer cells with the use of goat anti-mouse IgG magnetic beads (Dynal, Lake Success, N.Y.) coupled with mAbs to CD16 and CD56 (Becton Dickinson, San Jose, Calif.). CD4⁺ and CD8⁺ T cells were obtained from natural killer and CD14⁺-depleted cell suspensions by negative selection with the use of CD8⁺ and CD4⁺ magnetic beads, respectively (Dynal). CD8⁺ T cell suspensions were then twice depleted of CD28⁺ T cells with the use of goat anti-mouse IgG beads (Dynal) coupled with monoclonal anti-CD28 (Becton Dickinson). The purity of the CD4⁺ and CD8⁺ CD28⁻ T cell subsets was monitored by cytofluorographic analysis. All CD4⁺ and CD8⁺CD28⁻ T cell suspensions that were used in functional assays contained <2% CD16⁺CD56⁺ cells. CD4⁺ T cells were >98% positive for the CD4 and CD45RO markers. The population of CD8⁺CD28⁻ T cells contained >98% cells that were positive for CD8 and <2% CD28^{hi} cells.

Proliferation assays were done after two or three cycles of stimulation of human T cells with allogeneic CD2-depleted APCs or KG1 cells. Responding CD4⁺ T cells (1×10⁵ cells/well) were stimulated in triplicate with irradiated DCs (2.5×10⁴ cells/well), CD14⁺ monocytes (1×10⁵) or KG1 cells (5×10⁴) in the absence or presence of human CD8^{+CD}28⁻ T cells (1×10⁵ cells/well). Cultures were set up in 96-well trays in a total volume of 0.2 ml. In some experiments, monoclonal anti-ILT3, a mixture of mAbs to ILT4 and HLA class I (W6/32, ATCC) or exogenous rIL-2 (10 U/ml) were added at the start of incubation. After 48 h of incubation, the cultures were pulsed with [³H]thymidine and collected 18 h later. [³H]thymidine incorporation was determined by scintillation spectrometry.

Monocytes and DCs

Monocytes were obtained from PBMCs with the use of a Monocyte Negative Selection Kit (Dynal). Immature DCs were generated by culturing monocytes in 6-well plates at a concentration of 2×10⁶ cells per well for 7 days; GM-CSF (1000 U/ml, R&D Systems, Minneapolis, Minn.) and IL-4 (1000 U/ml, R&D Systems) were added on days 0, 2, 4, and 6 as described (44, 45). Immature DCs were CD14⁻CD11c⁺HLA-DR⁺, as shown by flow cytometry analysis.

Flow Cytometry

Flow cytometry studies were done with a FACScan (Becton Dickinson). CaliBRITE beads, from Becton Dickinson, were run under the FACSComp program to calibrate the instrument. Human CD4⁺ and CDB⁺CD28⁻ T cell subsets were defined by staining with phycoerythrin (PE)-conjugated monoclonal antibodies (mAbs) to CD3, CD28 and CD45RO; fluorescein isothiocyanate (FITC)-conjugated mAbs to CD4 and CD8; and mixtures of mAbs to FITC-CD3, PE-CD4, peridinine chlorophyll protein (PerCP)-CD8 and allophycocyanin-CD45 or FITC-CD3, PE-CD16-CD56, PerCP-CD19 and allophycocyanin-CD45 (Becton Dickinson). Other mAbs we used to stain CD8⁺ T cells included cychrome-CD38 and cychrome-HLA-DR (Becton Dickinson).

To study the expression of ILT3 or ILT4 and costimulatory molecules on normal APCs from peripheral blood or spleen cell suspensions, APCs were incubated with T_{S} cells for 18 h with or without CD40L-transfected D1.1 cells (see Results). The cells were then collected, washed and saturating amounts of mAbs to ILT3 or ILT4 were added (21, 22). After 30 min on ice, cells were washed twice, stained with PE-goat-anti-mouse or PE-goat-anti-rat (Caltech, Burlingame, Calif.), then washed twice again, incubated with mouse or rat IgG (as a blocking antibody, Vector Labs, Burlingame, Calif.), washed twice, then stained with mAbs to FITC-CD14 or cychrome-CD11c, FITC-HLA-DR and PerCP-CD3 (Pharmingen, San Diego, Calif.). In other samples, mAbs to PE-CD80 and PE-CD86 were added along with markers for monocytes and DCs. CD3⁺ T cells were gated-out and CD14⁺ monocytes or CD14⁻ CD11c⁺HLA-DR⁺ immature DCs were analyzed with CellQuest software on a G4 Apple Macintosh Computer. Annexin V and PI staining of target APCs was done as described (12). Five parameter analyses (forward scatter, side scatter and three fluorescence channels) were used for list mode data analysis. The FL3 channel was used as a fluorescence trigger and FL1 and FL2 as analysis parameters.

cDNA Microarray Profiling

Total RNA (1 pg) extracted from 1×10⁶-5×10⁶ KG1 cells or CD2-depleted normal APCs was radioactively labeled (with ³³P) by reverse transcriptase (Superscript, BRL, Rockville, Md.) and hybridized to a human UniGene Filter (GF211, Research Genetics, Huntsville, Ala.) at 42° C. for 16 h according to the manufacturer's instructions. After washing, the gene filter was exposed to a phosphorimaging screen and analyzed by Pathways 2 Software (Research Genetics, Huntsville, Ala.).

Semi-Quantitative RT-PCR

First-strand cDNA was synthesized from 1 μg of total RNA with a cDNA synthesis kit (Roche Diagnostic, Indianapolis, Ind.). The following primers were used in PCR reactions. ILT4: 5′ primer ACCCCCTGGACTCCTGATCAC; 3′ primer TGGAGTCTCTGCGTACCCTCC (expected size, 834 bp). ILT3: 5′ primer CAGACAGATGGACACTGAGG; 3′ primer AGAATCAGGTGACTCCCAAC (expected size, 320 bp). Primers for GADPH were as described (46). ILT3 and ILT4 PCR reactions were done at 30 cycles and GADPH PCR reactions were done at 23 or 24 cycles. PCR products were analyzed on agarose gel stained with ethidium bromide. RT-PCR products were quantified by digital imaging of the ethidium bromide agarose gel with a Kodak System 120; the images were analyzed on a computer with Kodak 1D Software (Kodak, Rochester, N.Y.). Values for ILT3 and ILT4 expression were normalized with the use of GAPDH expression values measured in the same cDNA dilutions. The normalized signals for each gene in untreated APCs were given a value of 1. Data were expressed as the mean±s.d. of all four different dilutions.

Construction of Retroviral Vectors Containing ILT3 and ILT4

Full-length ILT3 and ILT4 cDNAs were cloned from KG1 cells by RT-PCR into the pcDNA4/TO/myc-His vector (Invitrogen, Carlsbad, Calif.) in-frame with a COOH-terminal c-Myc tag. The Myc-tagged ILT3 and ILT4 inserts were subcloned into the BglII site of a green fluorescence protein (GFP)—retroviral vector called MIG (for MSCV-IRES-GFP) (47). The ILT3-MIG and ILT4-MIG inserts were completely sequenced from both strands to confirm that the correct sequence had been inserted. ILT3-MIG, ILT4-MIG or MIG alone (50 μg), PCL-eco (20 μg) and VSV-G (5 μg) were used to transfect 293T cells with the calcium phosphate method. Viral supernatants were collected 48 h after transfection and filtered through 0.45-μm membranes before use.

Generation and Characterization of KG1.ILT3 and KG1.ILT4 Cells

Retroviral transduction was via the centrifugal enhancement method (48). Briefly, KG1 cells were resuspended in viral supernatant (1-2 ml/10⁶ cells) with 8 μg/ml of polybrene (Sigma Chemical Co., St. Louis, Mo.), then centrifuged at 2500 g for 2 h at 30° C. Infected cells were resuspended in fresh Iscove's modified Eagle's medium and cultured overnight. After three consecutive spin-infections and overnight cultures, cells expressing high amounts of GFP were sorted with a FACStar Plus (Becton Dickinson). The sorted KG1.ILT3 and KG1.ILT4 cells, which were typically >95% GFP⁺, were used within 2-3 weeks. For each experiment, two or three independent transductants were tested.

Electrophoretic Mobility Shift Assay (EMSA)

Nuclear extracts were prepared and EMSAs were done as described (49). Double-stranded NF-κB oligomers (AGCTTCAGAGGGACTTTCCTCTGA) and double-stranded Sp1 oligomers (CCCTTGGTGGGGGCGGGGCCTAAGCTGCG) were used. KG1 cells incubated with CD4⁺ T_{H} cells were separated from the mixture with the use of CD34⁺ Dynal beads. For supershift assays, nuclear extracts prepared from CD4⁺ T_{H} cell-treated KG1 cells were incubated with antibodies to the NF-κB subunits p50 or p65 (Santa Cruz Biotechnology, Santa Cruz, Calif.) for 30 min at 4° C. before the labeled NF-κB probe was added.

Statistical Analysis

Analysis of the statistical differences between healthy controls and different groups of patients, with respect to the phenotype of CD₈⁺ T cells, were done with the use of one-way ANOVA tests followed by Scheffé criterion tests for multiple comparisons.

### C. Results

CD8⁺CD28⁻ T_{S} Cells Inhibit APC Allostimulatory Capacity

It has been previously shown that CD8⁺CD28⁻ T_{S} cells from allospecific and xenospecific TCLs inhibit CD4⁺ T_{H} cell proliferation in a dose-dependent manner (12, 13). Addition of either exogenous IL-2 or monoclonal anti-CD28 restored CD4⁺ T_{H} cell proliferation in the presence of CD8⁺CD28⁻ T_{S} cells, which indicates that the CD4⁺ T_{H} cells were rendered anergic (12, 15). CD8⁺CD28⁻ T_{S} cells recognize MHC class I alloantigens on APCs and render the APC unable to stimulate CD4⁺ T_{H} cell proliferation (12, 14).

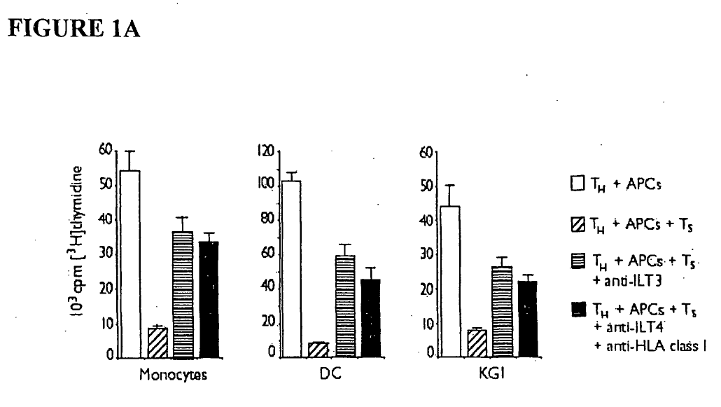

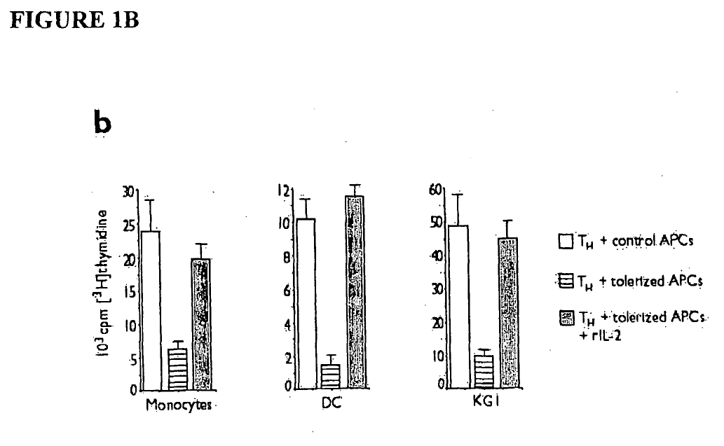

To determine the effect of CD8⁺CD28⁻ T_{S} cells on CD4⁺ T_{H} cell alloreactivity, 12 different TCLs were generated. For each TCL, T cells from a responder A were stimulated with CD2-depleted PBMCs from a stimulator B. After two rounds of allostimulation CD4⁺ T_{H} and CD8⁺CD28⁻ T_{S} cells from each TCL were purified, and CD4⁺ T_{H} cell alloreactivity was tested in 3-day proliferation assays. In these assays, CD4⁺ T_{H} cells from responder A or mixtures of CD4⁺ T_{H} and CD8⁺CD28⁻ T_{S} cells from responder A were stimulated with CD14⁺ monocytes or CD11c⁺ HLA-DR⁺CD14⁻ immature DCs from stimulator B. Immature DCs were generated from monocytes cultured with granulocyte-monocyte colony-stimulating factor (GM-CSF) and IL-4. When KG1 cells were used as stimulators to generate TCLs, KG1 cells were also used in the proliferation assay. CD8⁺CD28⁻ T_{S} cells isolated from each of these TCLs inhibited the blastogenic response of CD4⁺ T_{H} cells isolated from the same TCL to the specific stimulator by >80% (FIG. 1a).

Whether APCs exposed to CD8⁺CD28⁻ T_{S} cells become tolerogenic was investigated. Monocytes or DCs from the donor used for TCL priming were preincubated with allospecific CD8⁺CD28⁻ T_{S} cells. Similarly, KG1 cells were preincubated with KG1-primed CD8⁺CD28⁻ T_{S} cells. After 18 h, these conditioned APCs were γ-irradiated and used for stimulating CD4⁺ T_{H} cells in 3-day proliferation assays. CD8⁺CD28⁻ T_{S} cell-treated APCs induced little proliferation of allospecific CD4⁺T_{H} cells from the same TCL, whereas the proliferative responses of the same allospecific CD4⁺ T_{H} cells stimulated with untreated APCs were robust. Addition of recombinant IL-2 (rIL-2) to the allospecific CD4⁺ T_{H} cells restored CD4⁺ T_{H} responsiveness to CD8⁺CD28⁻ T_{S} cell-treated APCs (FIG. 1b). Hence, APCs pretreated with CD8⁺CD28⁻ T_{S} cells are poor inducers of CD4⁺ T_{H} cell activation; instead, these APCs induce CD4⁺ T_{H} cell anergy.

Anergizing APCs Express ILT3 and ILT4

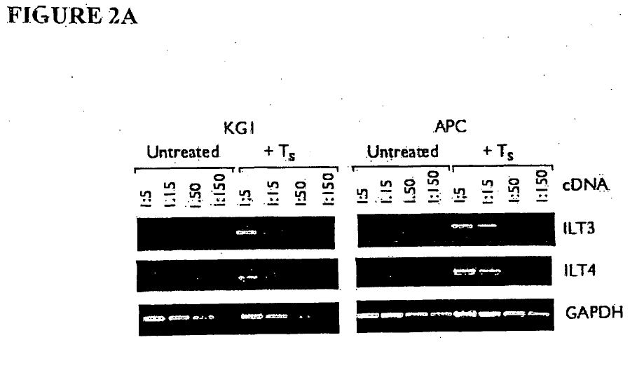

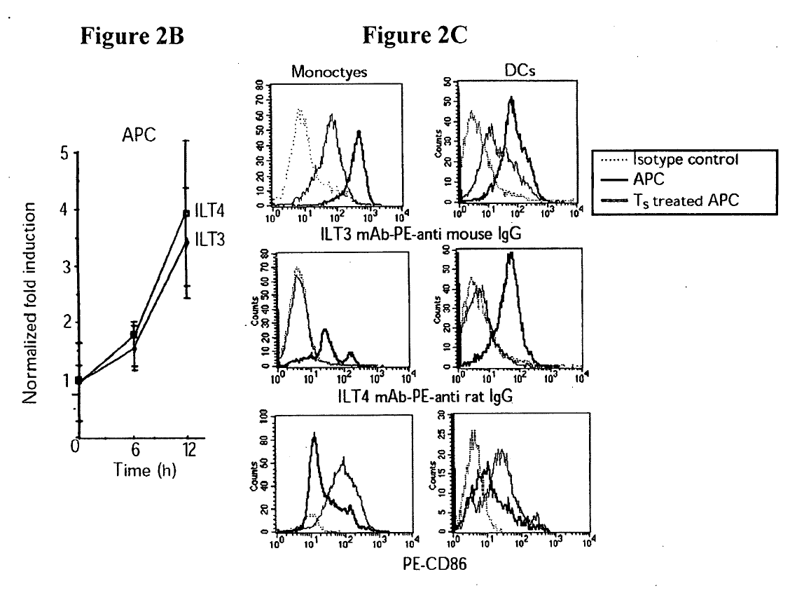

To identify the molecular changes associated with the acquisition of a tolerogenic phenotype the following steps were taken. First the effects of CD8⁺CD28⁻ T_{S} cell exposure on the transcriptional profile of KG1 cells and normal APCs were analyzed using a cDNA microarray that contained probes for 4454 randomly selected human genes. Expression profiling showed changes in both KG1 cells and normal APCs after treatment with CD8⁺CD28⁻ T_{S} cells. Among the changes confirmed by semi-quantitative reverse transcriptase-polymerase chain reaction (RT-PCR) was the up-regulation of mRNA encoding ILT3 and ILT4 in T_{S} cell-treated APCs and KG1 cells (FIGS. 2a and 2b). The inhibitory receptors ILT3 and ILT4 are selectively expressed by monocytes and DCs and are thought to play a physiological role in vivo by negatively regulating the activation of APCs (20-24). Thus, ILT3 and ILT4 protein expression on the surfaces of monocytes and DCs pretreated with allospecific CD8⁺CD28⁻ T_{S} cells was examined. Flow cytometry analysis showed that CD8⁺CD28⁻ T_{S} cells induced the up-regulation of ILT3 and ILT4 cell surface expression on both monocytes and DCs, whereas the expression of costimulatory molecules, such as CD86, was down-regulated (FIG. 2c).

These findings suggested that an inverse correlation exists between the up-regulation of ILT3 and ILT4 on APCs and the expression of costimulatory molecules. Because CD40 ligation resulted in up-regulation of CD80 and CD86 on APCs, the effect of CD8⁺CD28⁻ T_{S} cells on CD40 signaling in APCs was tested. We incubated CD8⁺CD28⁻ T_{S} cells (from responder A) with monocytes or DCs from the specific stimulator (B) in the presence or absence of CD40L-transfected D1.1 cells (25, 26). The same allospecific CD8⁺CD28⁻ T_{S} cells that induced up-regulation of ILT expression by APCs also suppressed the CD40L-mediated up-regulation of CD80 and CD86 on the same APC.

Next it was determined whether ILT3 and ILT4 were responsible for the reduced capacity of APCs to stimulate CD4⁺ T_{H} cell proliferation in the presence of CD8⁺CD28⁻ T_{S} cells. Monoclonal antibody (mAb) was added to ILT3 (24) or a mixture of mAbs to ILT4 (22) and HLA class I (the ligand for ILT4) to cultures containing allospecific CD4⁺ T_{H} cells, CD8⁺CD28⁻ T_{S} cells and the APCs used for priming. Neither mAb to ILT3 nor the mixture of mAbs to ILT4 and HLA class I had any effect on T_{S} or T_{H} cell proliferation in response to the specific stimulator. However, mAb to ILT3 or a mixture of mAbs to ILT4 and HLA class I-but neither mAbs to ILT4 nor HLA class I alone-reversed by 49±4% the inhibitory effect of T_{S} cells on CD4⁺ T_{H} cell proliferation in cultures containing mixtures of CD4⁺ T_{H} cells, CD8⁺CD28⁻ T_{S} cells and monocytes or DCs (FIG. 1a). These results indicated that the effect of CD8⁺CD28⁻ T_{S} cells on CD4⁺ T_{H} cell proliferation is mediated by the inhibitory receptors ILT3 and ILT4 on APCs.

Induction of T_{H} Cell Anergy

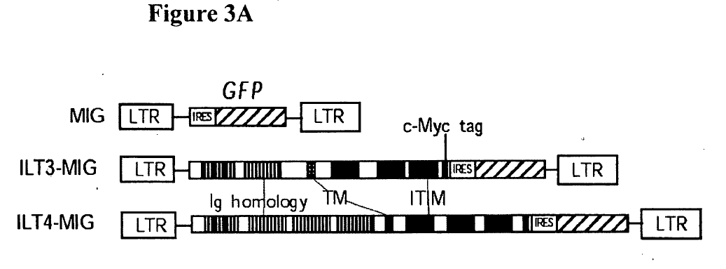

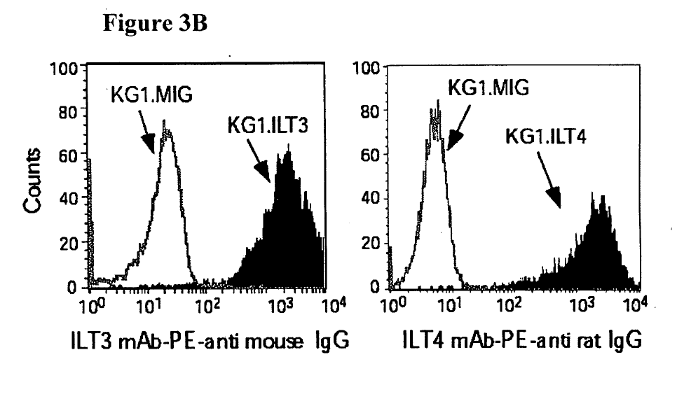

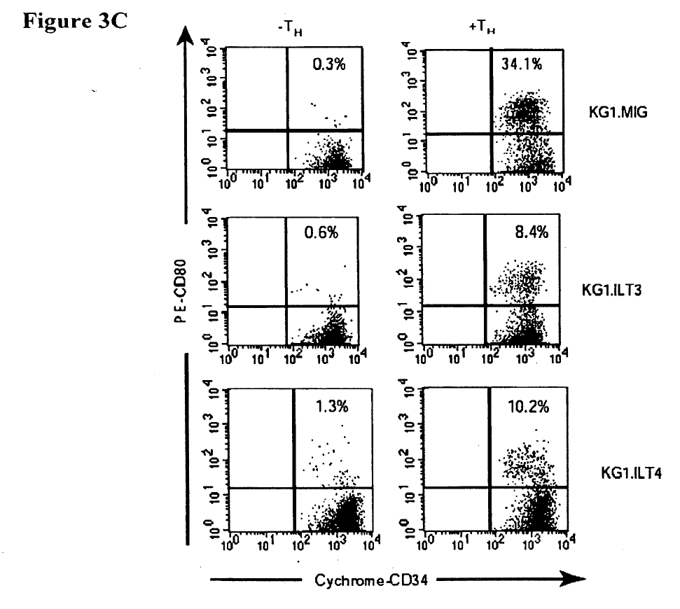

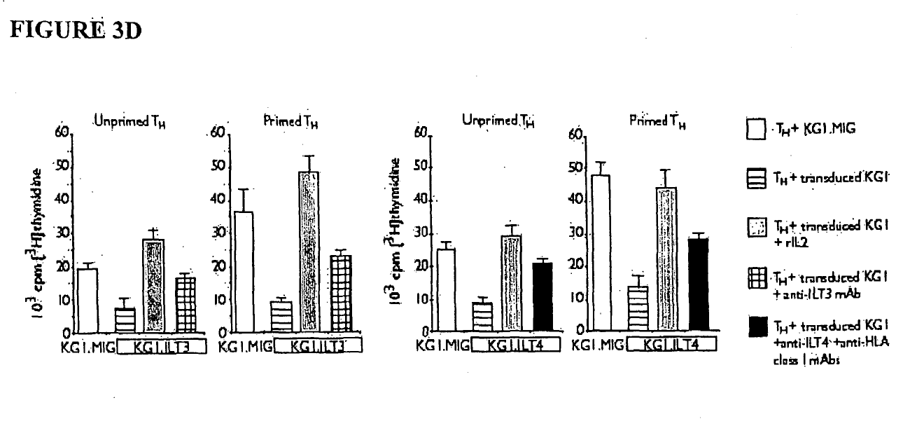

To further test the hypothesis that CD8⁺CD28⁻ T_{S} cell-induced up-regulation of ILT3 and ILT4 is responsible for the tolerogenic capacity acquired by APCs, ILT3 and ILT4 were overexpressed in KG1 cells as Myc fusion proteins via infection with recombinant retroviruses (FIG. 3a); ILT3- or ILT4-transduced KG1 cells-referred to hereafter as KG1.ILT3 and KG1.ILT4 cells, respectively-expressed high amounts of ILT3 or ILT4, as shown by flow cytometry (FIG. 3b) and confirmed by immunoblotting with anti-Myc. The basal expression of a variety of other markers—including HLA class I, HLA class II and costimulatory molecules—was similar in KG1 cells with empty vector alone (referred to hereafter as KG1.MIG cells) and in the KG1.ILT3 and KG1.ILT4 cells. In the presence of CD4⁺T_{H} cells, the percentage of CD80⁺ cells increased from basal amounts to 34.1% in the KG1.MIG cells (FIG. 3c). However, only 8.4% of the KG1.ILT3 cells and 10.2% of KG1.ILT4 cells expressed CD80 upon incubation with CD4⁺ T_{H} cells (FIG. 3c). Results obtained with three additional ILT3- and three ILT4-transduced KG1-independent clones—as well as four control KG1 clones transduced with vector alone—confirmed the finding that ILT3 and ILT4 overexpression interferes with the CD4⁺ T_{H} cell-induced up-regulation of CD80.

In addition, KG1.ILT3 and KG1.ILT4 cells elicited much less proliferation of unprimed and KG1-primed CD4⁺ T_{H} cells than KG1.MIG cells (FIG. 3d). Addition of rIL-2 to the proliferation assays restored CD4⁺ T_{H} cell proliferation, which supported the hypothesis that ILT3 or ILT4 overexpression renders KG1 cells tolerogenic (FIG. 3d). Addition of anti-ILT3 to cultures that contained CD4⁺ T_{H} and KG1.ILT3 cells restored the capacity of KG1.ILT3 cells to stimulate KG1-primed CD4⁺ T_{H} cells. Similarly, the mixture of mAbs to ILT4 and HLA class I restored the capacity of KG1.ILT4 cells to stimulate T_{H} cell proliferation (FIG. 3d). As overexpression of ILT3 and ILT4 conferred KG1 cells with a tolerogenic capacity, whereas mAbs to ILT3 and ILT4 partially blocked this effect, it appears that ILT3 and ILT4 have an immunoregulatory effect upon APCs.

NF-κB Activation is Inhibited in ILT3-Transduced APCs

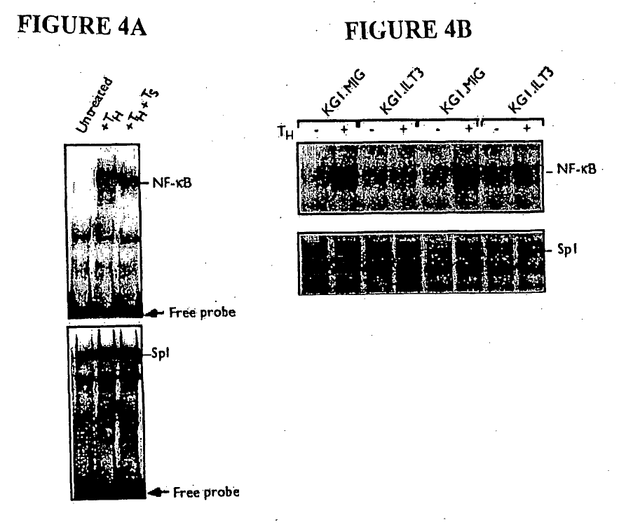

Because T_{S} cells inhibit NF-κB-mediated transcription of co-stimulatory molecules in APCs (17), it was tested whether constitutive expression of ILT3 in KG1 cells mimics some of the known effects of CD8⁺CD28⁻ T_{S} cells on APCs. NF-κB activation was measured by electrophoresis mobility-shift assays (EMSAs) (17) with the use of nuclear extracts from KG1 cells incubated for 6 hours with CD4⁺ T_{H} cells or mixtures of CD4⁺ T_{H} and CD8⁺CD28⁻ T_{S} cells. CD8^{CD}28⁻ T_{S} cells inhibited T_{H} cell-induced NF-κB activation in KG1 cells, yet these cells had no effect on the DNA-binding activity of the transcription factor Sp1 used as a nuclear extract control of the treated APC (FIG. 4a). Parallel studies done on KG1, KG1.MIG and KG1.ILT3 cells showed that ILT3 overexpression substantially reduced CD4⁺ T_{H} cell-induced NF-κB activation after 12 h of incubation but did not change the DNA-binding activity of Sp1 (FIG. 4b). Supershift experiments with antibodies specific for the p50 and p65 subunits of NF-κB showed that the observed bands represented p65-p50 complexes. Thus, ILT3-transduction led to the inhibition of T_{H} cell-induced NF-κB activation in KG1 cells.

ILT3 or ILT4 Expression on Donor APCs In Vivo

To determine whether up-regulation of ILT3 and ILT4 on APCs plays a role in vivo, the effect of CD8⁺CD28⁻ T cells from 15 heart allograft recipients (transplanted within 6-8 months of the experiment) on APCs from their respective cadaver donors were examined.

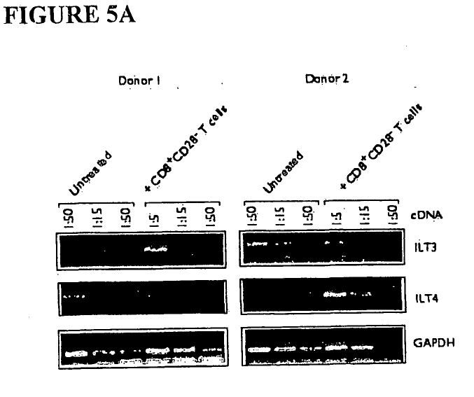

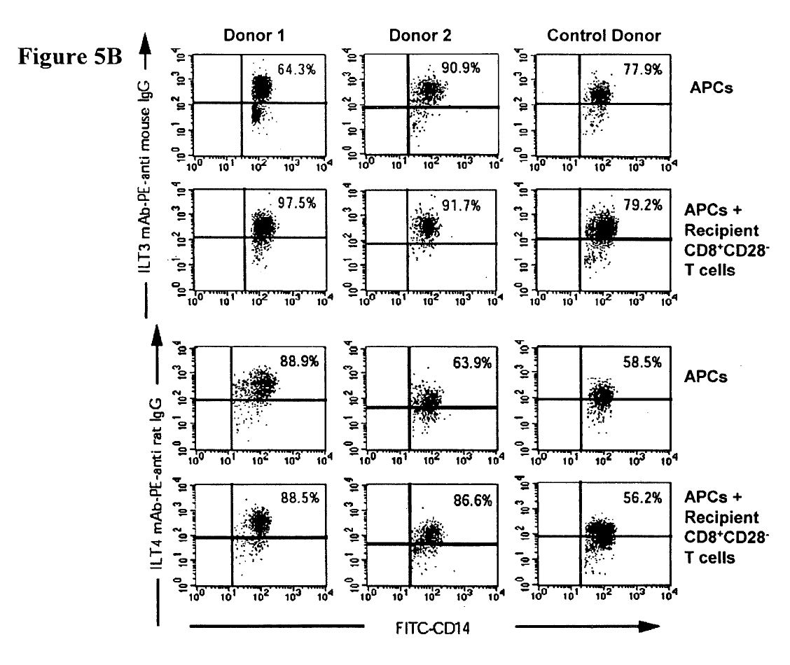

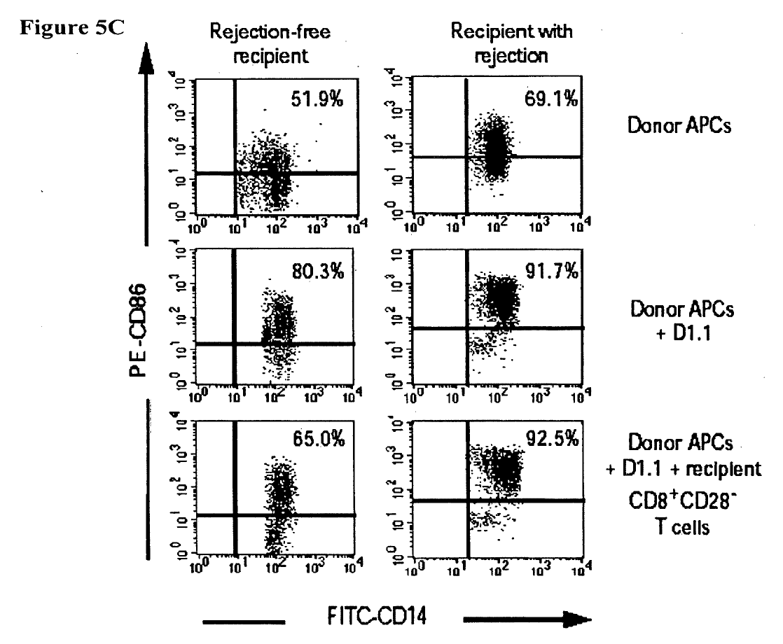

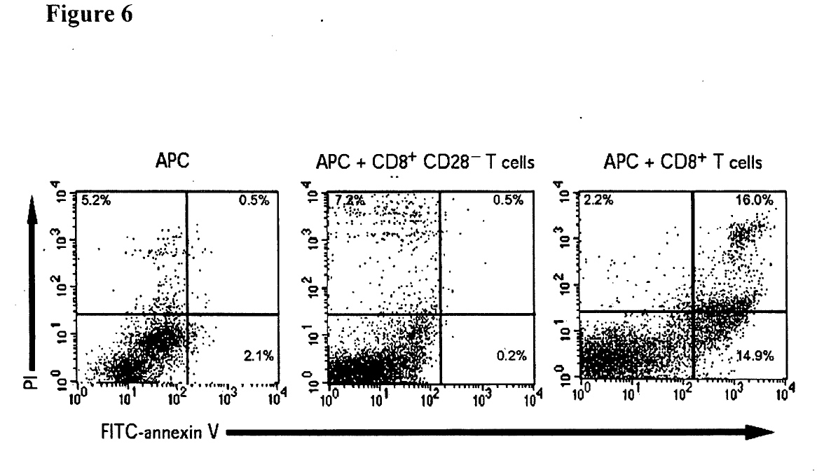

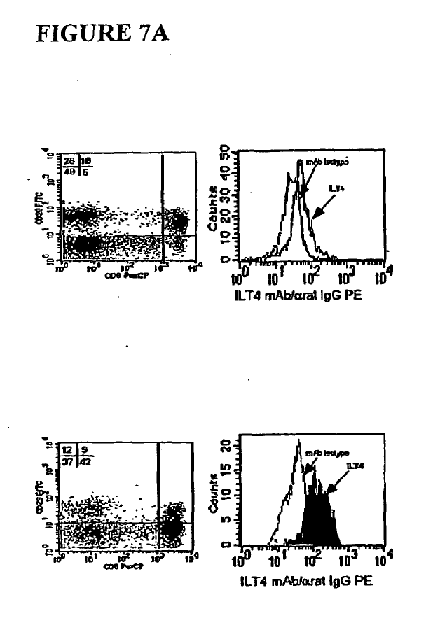

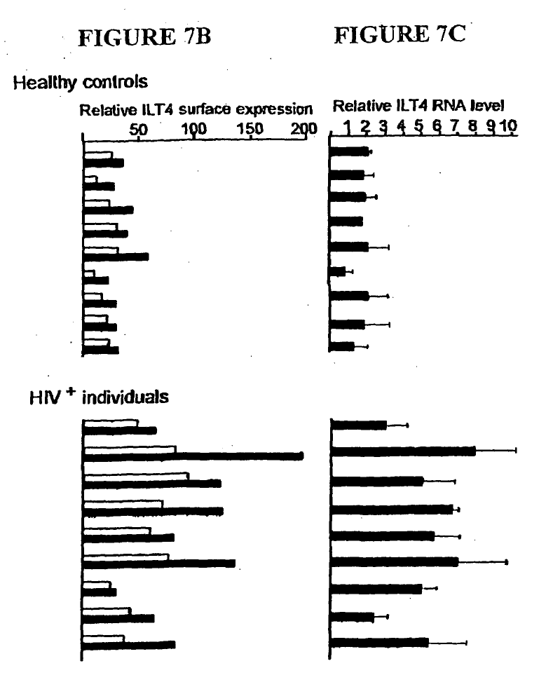

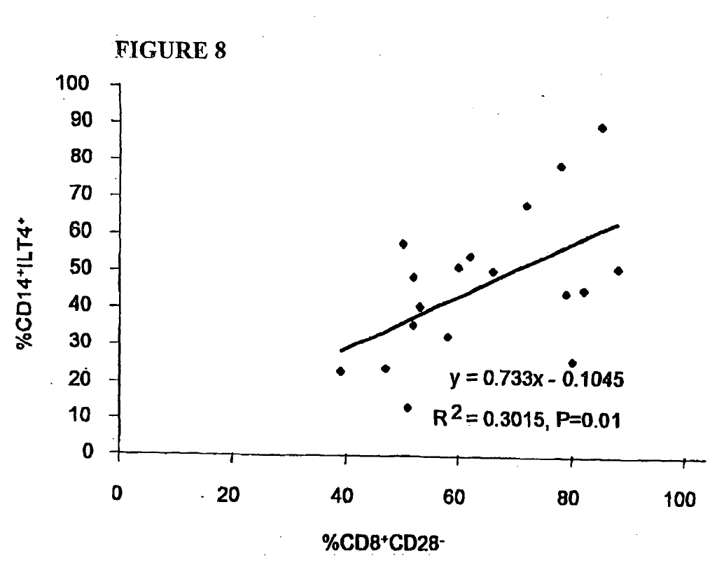

CD8⁺CD28⁻ T cells isolated from recipient's fresh peripheral blood were incubated for 18 h with CD2-depleted spleen cells from the heart donor or from a control individual who shared no HLA class I antigens with the transplant donor. Sufficient numbers of monocytes isolated from cryopreserved cadaver spleen that could be stained for ILT3 and ILT4 were obtained in only 10 of the 15 cases. However, expression of the inhibitory receptors was measured by the more sensitive RT-PCR method in all 15 cases. Five of the fifteen recipients studied had no acute rejection episode within the first 6-8 months after transplantation. CD8⁺CD28⁻ T cells from each of these patients induced the up-regulation of either ILT3 (two patients) or ILT4 (three patients) mRNA levels (FIG. 5a) and cell surface expression (FIG. 5b) on APCs from their corresponding donor. This effect was specific to the donor's HLA class I antigens, as CD8⁺CD28⁻ T cells from the rejection-free patients did not induce up-regulation of either ILT3 or ILT4 on control APCs displaying HLA antigens to which the recipient had not been exposed in vivo (FIG. 5b). CD8⁺CD28⁻ T cells from the remaining ten recipients had no effect on the level of ILT3 or ILT4 mRNA expressed by APCs. Nine of these ten heart transplant recipients experienced at least one episode of acute rejection (histological grade 2B or 3) within the first 6 months after transplantation. ILT3 and/or ILT4 was up-regulated in CD8⁺CD28⁻ T cells from all five patients without acute rejection induced, whereas CD8⁺CD28⁻ T cells from nine of ten patients with rejection had no such effect. This indicates that the capacity of T_{S} cells to induce up-regulation of ILT3 or ILT4 on donor monocytes is strongly associated with the absence of acute rejection (P<0.002).

The ability of recipient CD8⁺CD28⁻ T cells to inhibit CD40-triggered up-regulation of CD86 on donor APCs was also tested to determine whether CD8⁺CD28⁻ T cells primed in vivo with allogeneic HLA antigens behaved in a similar manner to T_{S} cells generated in vitro. Thus, five patients who had remained rejection-free after transplantation and five patients who had experienced two or three episodes of acute rejection within the first 8 months after transplantation were examined. CD8⁺CD28⁻ T cells from the rejection-free patients inhibited CD40L-triggered up-regulation of CD86 on donor APCs. In contrast, CD8⁺CD28⁻ T cells from patients with acute rejection episodes did not inhibit CD40 signaling (FIG. 5c). These findings suggested that similar to CD8⁺CD28⁻ T_{S} cells from an alloreactive TCL, CD8⁺CD28⁻ T cells from transplant recipients in quiescence induced the up-regulation of inhibitory receptors ILT3 and/or ILT4 and inhibited up-regulation of costimulatory molecules on APCs in an allospecific manner.

To establish whether T cells from transplant patients are cytotoxic to donor APCs, annexin V and propidium iodide (PI) were used to stain APCs incubated with CD8⁺CD28⁻ T cells and unfractionated CD8⁺ T cells from the recipients. Similar to quiescent patients, CD8⁺CD28⁻ T cells from patients with rejection showed no cytotoxic T cell activity in response to donor APCs. However, the nonfractionated CD8⁺ T cells (which contained both CD28⁻ and CD28⁺ T cells) from patients with rejection were capable of killing donor APCs. Unfractionated CD8⁺ T cells from quiescent patients showed no cytotoxic activity. This suggests that CD8⁺CD28⁺ T cells from allosensitized recipients act as effectors of allograft rejection (FIG. 6).

Annexin V and PI staining of CD20⁺ and CD14⁺ splenocytes from the transplant donor incubated with or without CD8⁺CD28⁻ T cells or unfractionated CD8⁺ T cells from the corresponding recipient.

Both CD8⁺CD28⁻ and the CD8⁺CD28⁺ T cells from transplant recipients and healthy controls were further characterized with respect to the frequency of CD38⁺, CD45RO⁺ and HLA-DR⁺ cells within the CD8⁺ subset. The percentage of CD8⁺CD28⁻ T cells was higher in heart recipients compared to healthy controls (P<0.01); this concurred with published data on liver transplant recipients (27). The frequency of CD8⁺CD28⁻ T cells that were expressing CD38, CD45Ro and HLA-DR was also higher in transplant recipients compared to controls (P<0.01), yet there was no difference between patients with or without rejection. The frequency of CD38- and CD45RO-expressing T cells within the CD8⁺CD28⁺ subset was not significantly different between patients that had or had not undergone rejection or healthy controls. However, transplant recipients showed a higher frequency of CD8⁺CD28⁺HLA-DR⁺ T cells compared to the controls (P<0.01) (Table 1). These results indicate that although compared to healthy controls, transplant patients show an expansion of T cells with memory and activation markers, the phenotype of CD8⁺CD28⁻ or CD8⁺CD28⁺ T cells from patients that had or had not undergone rejection does not differ with respect to these markers.

TABLE 1
Phenotypic Characterization of CD8+ T Cells
From Transplant Patients and Healthy Controls

| | Transplant patients | | |
|---|---|---|---|
| Cell subsets | Controls (%) a | No rejection (%) b | Rejection (%) c |
| CD8+ | 20.70 ± 4.95 | 20.17 ± 1.94 | 20.89 ± 4.04 |
| CD8+CD28− | 22.20 ± 6.79 | 63.33 ± 6.35 | 60.89 ± 4.91 |
| CD8+CD28+ | 77.30 ± 6.91 | 38.33 ± 5.13 | 39.22 ± 4.94 |
| CD8+CD28−CD38+ | 14.20 ± 4.31 | 36.67 ± 3.93 | 33.11 ± 2.57 |
| CD8+CD28+CD38+ | 4.05 ± 1.39 | 6.80 ± 1.94 | 5.67 ± 2.12 |
| CD8+CD28−CD45RO+ | 12.20 ± 2.63 | 28.83 ± 4.17 | 25.67 ± 3.57 |
| CD8+CD28+CD45RO+ | 37.20 ± 4.73 | 39.67 ± 5.57 | 35.44 ± 4.07 |
| CD8+CD28−HLA−DR+ | 7.19 ± 3.48 | 36.24 ± 3.99 | 38.56 ± 5.05 |
| CD8+CD28+HLA−DR+ | 10.51 ± 5.44 | 20.17 ± 5.00 | 23.78 ± 3.60 |

a Data are mean ± S.D. from 20 individuals
b Data are mean ± S.D. from 6 individuals
c Data are mean ± S.D. from 9 individuals

### D. Discussion

ILT3 and ILT4 are up-regulated in APCs after exposure to CD8⁺CD28⁻ T_{S} cells and are essential to the tolerogenic phenotype acquired by APCs. The CD4⁺ T_{H} cell unresponsiveness induced by CD8⁺CD28⁻ T_{S} cell-treated APCs is characteristic of T cell anergy, as the loss of CD4⁺ T_{H} cell proliferative capacity can be reversed by the addition of exogenous IL-2 (28). TCR-triggering (signal 1) in the absence of costimulation (signal 2) results in T cell anergy (29). Tolerogenic APCs showed decreased amounts of costimulatory molecules in conjunction with increased ILT3 and ILT4 expression.

Although it has been speculated that ITIM-bearing ILTs may control DC antigen-presenting functions, co-stimulation and cytokine production, their physiological significance was unknown. Now it has been demonstrated that up-regulation of ILT3 and ILT4 renders monocytes and DCs tolerogenic. The finding that overexpression of ILT3 was associated with inhibition of NF-κB activation shows that, in the presence of CD8⁺CD28⁻ T_{S} cells, APCs have a reduced capacity to transcribe NF-κB-dependent costimulatory molecules (17). Although it is not clear how ILT3 and ILT4 overexpression interferes with CD40 signaling, it is possible that these receptors act through SHP phosphatases to modulate IκB phosphorylation and degradation, thus affecting NF-κB activation. This would inhibit the transcription of NF-κB-dependent genes that encode co-stimulatory molecules in DCs, thus promoting their capacity to induce CD4⁺ T_{H} cell anergy (22-24).

Other strategies that inhibit the expression of co-stimulatory molecules, such as treatment with corticosteroids (30), vitamin D3 (31) and culture with a suboptimal dose of GM-CSF (32) can also successfully generate tolerogenic APCs (33). It remains to be seen, however, whether up-regulation of ILT3 and ILT4 also occurs in such APCs with tolerogenic activity.

In vivo evidence that ILT3 and ILT4 expression is associated with an anergizing APC phenotype was provided by a study of transplant patients. CD8⁺CD28⁻ T cells from quiescent patients induced up-regulation of ILT3 or ILT4 on donor monocytes and inhibited CD40-signaling (25).

CD4⁺ T_{H} cells from transplant recipients recognize MHC alloantigens directly on donor APCs (direct pathway of allorecognition) or indirectly on self-APCs that have captured and processed antigens from dying graft cells (indirect pathway of allorecognition) (33). In vitro studies have shown that CD8⁺CD28⁻ T_{S} cells primed with allogeneic APCs inhibit the direct pathway (12), whereas CD8⁺CD28⁻ T_{S} cells primed with self-APCs pulsed with allopeptides inhibit the indirect allorecognition pathway (14). The finding that CD8⁺CD28⁻ T cells from quiescent transplant recipients induced the up-regulation of ILT3 or ILT4 and inhibited CD40-signaling by donor APCs indicates the presence of a population of allospecific T_{S} cells that may inhibit the direct recognition pathway involved in allograft rejection. It is possible that suppression of the direct recognition pathway can be achieved by treating the organ with agents that induce the up-regulation of ILT3 and ILT4 on donor DCs before transplantation. After transplantation, donor DCs that are overexpressing ILT3 and ILT4 may induce T_{H} cell anergy in situ or in the draining lymph nodes. Apoptotic donor DCs will be captured and processed by recipient DCs in the lymph nodes. Recipient allopeptide-specific T cells may be cross-tolerized by these autologous DCs that present alloantigens in the absence of inflammatory cytokines. Alternatively, the indirect pathway could also be suppressed by inducing the overexpression of ILT3 and ILT4 on autologous (recipient) DCs that are generated and allopeptide-pulsed ex vivo.

Several other ITIM-bearing receptors, such as mouse PD-1, contribute to immune regulation, as mice that are homozygous for a disrupted PD-1 gene also develop autoimmune diseases (34). Other autoimmune disorders have been linked to single-point mutations in SHP-1 (35).

There is increasing evidence that DCs are central both to the activation and to the suppression of the immune response (35-39). The finding that CD8⁺CD28⁻ T_{S} cells induce up-regulation of ILT3 and ILT4 is critical to the tolerogenic properties acquired by APCs and supports the concept that the functional state of an APC dictates the outcome of an immune response (40, 41).

Although the focus in the description above has been focused on CD8⁺CD28⁻ T cell-mediated suppression, T_{R} cells with other phenotypes and/or their cytokines may act via a similar mechanism. This idea is supported by recent experiments that showed that within 20 h of incubation with IL-10, the expression of ILT3 and ILT4 on the membranes of human monocytes and DCs was up-regulated, whereas CD86 was down-regulated. In contrast to other T_{R} cells from the CD8⁺ and CD4⁺ subsets that exert their inhibitory effects via IL-10, CD8⁺CD28⁻ T_{S} cells do not produce IL-10 (2, 10, 11). Therefore, the up-regulation of ILT3 and ILT4 induced in APCs by CD8⁺CD28⁻ T_{S} cells is not unique to these cells, but more likely is a feature that is shared with other inhibitors of antigen-specific T cell responses.

The plasticity of DCs and their capacity to polarize T cells toward functionally distinct subsets seems to be central to the regulation of the immune response (42). The data we present here suggest that the modulation of ILT3 or ILT4 expression on APCs may permit the development of tolerogenic or immunogenic vaccines.

### E. Supplemental Experimental Discussion

The upregulation of ILT3 and ILT4 induced in monocytes and dendritic cells by CD8⁺CD28⁻ T_{S} is not a unique property of these cells but a feature shared with other inhibitors of antigen specific T cell responses. For example, treatment of APC with IL-10, vitamin D3 analogs or CD4⁺CD25⁺ regulatory T cells also induces upregulation of ILT3-ILT4 rendering these APC tolerogenic.

This finding implies the following:

(1) Agents that have the capacity of rendering dendritic cells tolerogenic can be easily identified by testing their ILT3-ILT4 enhancing activity.
(2) The discovery of methods for generating tolerogenic APC has immediate application in clinical organ transplantation. “Passenger” APC of donor origin are always present in solid organ transplants. These “passenger” APC are the imitators of allograft recognition and rejection, stimulating recipient T cell responses. Pre-treatment of the transplant with tolerogenic agents will not only prevent the “passenger” APC from eliciting T cell reactivity in the host, but by inducing ILT3 and ILT4 upregulation they render these APC capable to anergize T cells with the corresponding TCR. Hence, T cells which recognize Major Histocompatibility Complex (MHC) antigens on donor APC will become tolerant. This phenomenon can be defined as blocking of the direct recognition pathway.

Apoptotic donor dendritic cells (DC) will be captured and processed by recipient DC in the lymph nodes. Recipient T cells recognize donor MHC/peptide complexes, expressed on the membrane of immature host dendritic cells, will render T cells anergic blocking the indirect pathway of allorecognition. Therefore, specific tolerance to organ allografts can be induced by pre-treating the graft with tolerogenic agents.

(3) Tolerogenic agents have the potential of increasing substantially the availability of donors for bone marrow transplantation.

Currently, bone marrow or umbilical cord stem cell transplants are performed only when there is complete matching for HLA-A, B, DR and DQ antigens between the recipient and the donor. In the absence of HLA-identical donor from the family (sibling) the likelihood of finding a suitable donor is less than 1 in 1,000,000. For this reason, the cost of HLA-typing in search of a donor, stem cell preservation and transplantation is outrageously high. Furthermore, graft-versus-host disease will occur in about 50% of the recipients, leading to high mortality.

The discovery that overexpression of ILT3 and ILT4 on APC renders these cells capable to anergize T cells implies the possibility of using HLA-mismatched stem cell donors for transplanting recipients pre-treated by use of tolerogenic agents. If host APC become tolerogenic, donor T cells will be anergized rather than activated, avoiding the Graft versus Host Disease.

By analogy Host versus Graft reactions in solid organ transplantation could be avoided by treatment of recipient with tolerogenic DC from the donor or from an unrelated individual sharing HLA antigens with the donor.

(4) Patients with AIDS have an increased number of CD4+ and CD8+ T cells which upon stimulation (with mitogens) produce IL-10 and TNF-alpha (J. Acquir Immune Defic Synd 20001, Dec. 15, 28(5) 429-438). These two cytokines increase the expression of ILT3 and ILT4 on monocytes and dendritic cells. Not only do patients with AIDS display a greater than 10 fold increase in the level of ILT4 expression on APC, but their sera also increase ILT3 and ILT4 expression on APC from healthy blood donors.

It results that APC from AIDS patients, which overexpress ILT3 and ILT4, present peptides derived from the processing of pathogens in a tolerogenic form. To prevent T cell tolerization by self-APC, ILT3 and ILT4 interaction with patient's T cells must be blocked. Receptor blockade is generally accomplished either by treatment with “blocking” antibodies or by treatment with a soluble form of the ligand. While the ligand for ILT3 is not known as yet, the ligand for ILT4 is known to be HLA-A, B and G. Hence, treatment of patients with soluble HLA-G may prevent the interaction between the T cell surface ligand of ILT4 with the ILT4-receptor on APC thus preventing the transduction of inhibitory signals.

(5) Another field of clinical immunology that may greatly benefit from our discovery resides in the treatment of autoimmune diseases.

The current dogma is that autoimmunity results from cross-priming the patients' T cells by dendritic cells which present tissue or organ-specific peptides, derived from cells undergoing necrosis under inflammatory conditions. To prevent progression of the autoimmune response it may be sufficient to treat the patient with autologous dendritic cells that have processed ex vivo apoptotic cells of the target organ (for example, pancreatic islets, thyroid cells, etc). Treatment with vitamin D3, IL-10 or other tolerogenic agents may render these antigen-pulsed dendritic cells tolerogenic. This will permit blocking of the autoimmune disease.

Direct administration of tolerogenic agents or ex vivo manipulation of patients' dendritic cells may accomplish this purpose.

(6) It has been hypothesized that progression of malignancies is caused by the inefficiency of the immune response against tumor-specific peptides/MHC class I complexes. Notoriously, numerous tumors display a decrease in the level of expression of some (but not all MHC antigens) and may thus escape recognition by antigen-specific T cells. It is conceivable that in the absence of inflammatory cytokines, patients' APC will be unable to cross prime the T cell response against the tumor. To create conditions that optimize “licensing” of APC to stimulate an immune response versus tolerization of APC that will inhibit an immune response, it may be necessary to block the capacity of patients' APC to transcribe ILT3-ILT4.

This may be accomplished by depletion of CD8+CD28CD27+ T suppressor cells and of CD4+CD25+ T regulatory cells or by specific inhibition of ILT3 and ILT4 transcription.

In conclusion, the discovery of the inhibitory function of ILT3 and ILT4 receptors concerns the central control mechanisms of the immune response which must be inhibited to induce specific tolerance in transplantation and autoimmune diseases and augmented in AIDS and Cancer.

Modulation of ILT3 or ILT4 expression on APC may permit the development of tolerogenic or immunogenic vaccines. Ex vivo manipulation of dendritic cells to express high levels of ILT3-ILT4 or conversely, to express low levels of these molecules will result in the generation of APC which elicit tolerance or immunity, respectively.

### Second Series of Experiments

Study of ILT3 and ILT4 in monocytes from HIV-infected patients and non-infected individuals.

### A. Introduction

It has been shown that the number of CD8⁺CD28⁻ T cells increases massively during HIV infection and progression to AIDS (21-23). These cells have impaired cytolytic function which is associated with persistent expression of CD27 (22) and inhibitory NK receptors (iNKRs) (24-26). The fact that HIV infected patients have expanded CD8⁺ CD28⁻ T cell population provided the opportunity to examine the consequences of this expansion on APC phenotype and function.

### B. Results

First, the relationship between CD8⁺ CD28⁻ T cells and ILT3/ILT4 expression on monocytes from immunologically deficient, HIV infected patients were analyzed. This study included a population of 18 HIV-infected and 15 uninfected healthy, individuals. Phenotypic characterization of peripheral blood T lymphocytes showed that HIV-infected individuals had a significantly higher frequency of CD8⁺ CD28⁻ (FIG. 7a), CD28⁺ CD28⁻ CD27+, and CD8⁺ CD28⁻ CD94⁺ T cells compared to non-infected individuals (Table 2), in agreement with other investigators data (21, 24-26). The percentage of monocytes expressing ILT4 was also significantly increased in patients (FIG. 7a, 7b, and Table 2). Furthermore, the mean channel of fluorescence intensity was significantly higher (103.11±71.13) in HIV infected than in healthy non-infected individuals (48.10±24.27) (p<0.006), as illustrated in FIGS. 7a and 7b.

TABLE 2
Phenotypic Characteristics of T Cells and Monocytes
from HIV-infected and Non-Infected Individuals

| | Healthly Controls | HIV+ Patients | |
|---|---|---|---|
| Cell Type | (Mean ± S.D.) | (Mean ± S.D.) | P-value |
| % CD45+CD3+ | 72.40 ± 10.26 | 73.44 ± 11.57 | N.S. |
| % CD45+CD3+CD4+ | 51.20 ± 8.16 | 15.88 ± 6.64 | 0.0001 |
| % CD45+CD3+CD8+ | 19.00 ± 3.80 | 54.11 ± 12.98 | 0.0001 |
| % CD8+CD28− | 24.60 ± 12.42 | 63.22 ± 16.36 | 0.0006 |
| % CD8+CD28−CD27+ | 7.66 ± 3.72 | 21.05 ± 10.41 | 0.0049 |
| % CD8+CD28−CD94+ | 11.56 ± 7.30 | 27.37 ± 8.72 | 0.0029 |
| % CD14+ILT4+ | 20.20 ± 11.22 | 46.88 ± 20.03 | 0.0007 |

Consistent with these results, ILT4 mRNA level measured by semiquantitative RT-PCR in CD14⁺ cells was 3-5 fold higher in HIV infected individuals than in healthy controls (FIG. 7c). There was a direct correlation between the frequency of CD8+CD28⁻ T cells and the percentage of ILT4⁺ monocytes in the population of HIV infected patients (FIG. 8), suggesting the possibility that these two phenomena are inter-related. Analysis of ILT3 cell surface expression and mRNA levels in monocytes from patients and controls showed no quantitative differences.

To determine whether increased expression of ILT4 in CD14⁺ cells from HIV infected patients is associated with impaired APC function the MLC stimulatory capacity of patients' monocytes were compared with that of normal controls using as responders PBMC from healthy blood donors. Monocytes from HIV infected individuals, failed to induce T cell alloreactivity. Since the expression of HLA class II antigens and CD86 molecules on APC from HIV and control individuals did not differ, it appears that the impaired MLC stimulatory capacity of patients' monocytes is related to upregulation of ILT4.

### C. Discussion

To study the biological relevance of the ILT3 and ILT4 molecules induced by T_{S} in APC, the expression of these inhibitory receptors were analyzed in HIV-infected patients, known to exhibit an increased population of CD8⁺CD28⁻ T cells (21-23). The frequency of CD14⁺ monocytes expressing ILT4, as well as the mean channel of fluorescence was significantly higher in HIV-infected patients than in healthy controls.

Although it has not been directly determined that the noncytotoxic CD8⁺ CD28⁻ T cells seen in patients with HIV infection (21-23) have suppressor function and are responsible for the increased level of ILT4 expression on monocytes, there is found a direct correlation between the frequency of the CD8⁺ CD28⁻ T cells and that of CD14⁺ ILT4⁺ cells. This correlation suggests the possibility that Ts, induced during the immune response to viral or microbial antigens, may be responsible for upregulation of ILT4 expression in HIV-infected patients.

The increased expression of ILT4 in monocytes from HIV-infected patients may be responsible for their lack of allostimulatory activity. This suggests that ILT4⁺ monocytes from HIV infected individuals may inhibit Th activation and proliferation.

### Third Series of Experiments

### A. Introduction

Patients infected with Hepatitis C Virus (HCV) have an impaired response against HCV antigens while maintaining immune competence for other antigens. Although some patients exhibit acute self-limited infection the majority of them (70%) display persistent infection and chronic hepatitis with a strong risk for the development of hepatocellular carcinoma. T cell responses against viral antigens are vigorous in individuals who have cleared HCV after acute infection or after treatment with alpha interferon, while patients who fail to respond to therapy exhibit poor reactivity.

### B. Results

The impairment of T cell reactivity in patients with chronic infection may be secondary to virus induced alterations of APC function. The level of expression of the inhibitory receptors ILT3 and ILT4 were tested on monocytes from 13 patients with chronic infection and from 8 patients that have resolved infection (as determined by a negative PCR test of their serum). In all patients with chronic infection the level of ILT3 and ILT4 expression was significantly higher (more than 50% positive monocytes) than in patients who have resolved infection or healthy controls (less than 25% positive monocytes) (P<0001). Furthermore, monocytes from patients with chronic infection displayed low allostimulatory capacity in conjunction with high ILT3/ILT4 expression indicating impaired antigen presenting function. Their stimulatory capacity, however, was restored in cultures containing anti-ILT3 and ILT4 antibodies.

### C. Conclusion

These data suggest that quantitation of ILT4 expression on patients' monocytes provides an excellent parameter for assessing their immunologic competence. Furthermore, it appears that blockade of inhibitory receptors ILT3 and ILT4 on APC is required for restoring APC function and implicitly T cell immunity in patients with chronic HCV infection.

### REFERENCES

### References for the Background of the Invention and First Series of Experiments

1. Sakaguchi, S. Regulatory T cells: key controllers of immunologic self-tolerance. Cell 101, 455-458 (2000).
2. Roncarolo, M-G., Levings, M. K. & Traversari, C. Differentiation of T regulatory cells by immature dendritic cells. J. Exp. Med. 193, 5-9 (2001).
3. Shevach, E. M. Certified professionals: CD4(+)CD25(+) suppressor T cells. J. Exp. Med. 193, 41-46 (2001).
4. Shevach, E. M. Regulatory T cells in autoimmunity. Annu. Rev. Immunol. 18, 423-449 (2000).
5. Levings, M. K., Sangregorio, R. & Roncarolo, M. G. Human CD25(+)CD4(+) T regulatory cells suppress naive and memory T cell proliferation and can be expanded in vitro without loss of function. J. Exp. Med. 193, 1295-1302 (2001).
6. Takahashi, T. et al. Immunologic self-tolerance maintained by CD25⁺CD4⁺ regulatory T cells constitutively expressing cytotoxic T lymphocyte-associated antigen 4. J. Exp. Med. 192, 303-310 (2000).
7. Berzofsky, J. A., Ahlers, J. D. & Belayokov, J. M. Strategies for designing and optimizing new generation vaccines. Nature Rev. Immunol. 1, 209-219 (2001).
8. Read, S., Malmstrom, V. & Powrie, F. Cytotoxic lymphocyte-associated antigen 4 plays an essential role in the function of CD25(+)CD4(+) regulatory cells that control intestinal inflammation. J. Exp. Med. 192, 295-302 (2000).
9. Jonuleit, H. et al. Identification and functional characterization of human CD4(+)CD25(+) T cells with regulatory properties isolated from peripheral blood. J. Exp. Med. 193,1285-1294 (2001).
10. Groux, H. et al. A CD4⁺ T-cell subset inhibits antigen-specific T-cell responses and prevents colitis. Nature 389, 737-742 (1997).
11. Dhodapkar, M. V., Steinman, R. M., Krasovsky, J., Munz, C. & Bhardwaj, N. Antigen-specific inhibition of effector T cell function in humans after injection of immature dendritic cells. J. Exp. Med. 193, 233-238 (2001).
12. Liu, Z., Tugulea, S., Cortesini, R. & Suciu-Foca, N. Specific suppression of T helper alloreactivity by allo-MHC class I-restricted CD8⁺ CD28⁻ T cells. Int. Immunol. 10, 775-783 (1998).
13. Ciubotariu, R. et al. Specific suppression of human CD4⁺ Th cell responses to pig MHC antigens by CD8⁺ CD28⁻ regulatory T cells. J. Immunol. 161, 5193-5202 (1998).
14. Jiang, S. et al. Induction of MHC-class I restricted human suppressor T cells by peptide priming in vitro. Hum. Immunol. 59, 690-699 (1998).
15. Colovai, A. I. et al. Induction of xenoreactive CD4⁺ T-cell anergy by suppressor CD8⁺ CD28⁻ T cells. Transplantation 69, 1304-1310 (2000).
16. Liu, Z., Tugulea, S., Cortesini, R., Lederman, S. & Suciu-Foca, N. Inhibition of CD40 signaling pathway in antigen presenting cells by T suppressor cells. Hum. Immunol. 60, 568-574 (1999).
17. Li, J. et al. T suppressor lymphocytes inhibit NFκB-mediated transcription of CD86 gene in APC. J. Immunol. 163, 6386-6392 (1999).
18. Damle, N. K., Mohagheghpour N., Hansen J. A. & Engleman E. G. Alloantigen-specific cytotoxic and suppressor T lymphocytes are derived from phenotypically distinct precursors. J. Immunol. 131, 2296-300 (1983).
19. St. Louis, D. C. et al. Evidence for distinct intracellular signaling pathways in CD34⁺ progenitor to dendritic cell differentiation from a human cell line model. J. Immunol. 162, 3237-3248 (1999).
20. Colonna, M., Nakajima, H., Navarro, F. & Lopez-Botet, M. A novel family of Ig-like receptors for HLA class I molecules that modulate function of lymphoid and myeloid cells. J. Leukoc. Biol. 66, 375-381 (1999).
21. Colonna, M., Nakajima, H. & Cella, M. A family of inhibitory and activating Ig-like receptors that modulate function of lymphoid and myeloid cells. Semin. Iunmunol. 12, 121-127 (2000).
22. Colonna, M. et al. Cutting edge: human myelomonocytic cells express an inhibitory receptor for classical and nonclassical MHC class I molecules. J. Immunol. 160, 3096-3100 (1998).
23. Ravetch, J. V. & Lanier, L. Immune inhibitory receptors. Science 290, 84-88 (2000).
24. Cella, M. et al. A novel inhibitory receptor (ILT3) expressed on monocytes, macrophages, and dendritic cells involved in antigen processing. J. Exp. Med. 185, 1743-1751 (1997).
25. Ciubotariu, R. et al. Detection of T suppressor cells in patients with organ allografts. Hum. Immunol. 62, 15-20 (2001).
26. Yellin, M. J. et al. T lymphocyte T cell-B cell-activating molecule/CD40-L molecules induce normal B cells or chronic lymphocytic leukemia B cells to express CD80 (B7/BB-1) and enhance their costimulatory activity. J. Immunol. 153, 666-674 (1994).
27. Garcia-Alonso, A. M. et al. CD28 expression on peripheral blood T lymphocytes after orthotopic liver transplant: upregulation in acute rejection. Hum. Immunol. 53, 64-72 (1997).
28. Schwartz, R. H. Models of T cell anergy: is there a common molecular mechanism? J. Exp. Med. 184, 1-8 (1996).
29. Jenkins, M. K. & Schwartz, R. H. Antigen presentation by chemically modified splenocytes induces antigen-specific T cell unresponsiveness in vitro and in vivo. J. Exp. Med. 165, 302-319 (1987).
30. Rea, D. et al. Glucocorticoids transform CD40-triggering of dendritic cells into an alternative activation pathway resulting in antigen-presenting cells that secrete IL-10. Blood 95, 3162-3167 (2000).
31. Penna, G. & Adorini, L. 1α,25-dihydroxyvitamin D3 inhibits differentiation, maturation, activation, and survival of dendritic cells leading to impaired alloreactive T cell activation. J. Immunol. 164, 2405-2411 (2000).
32. Lutz, M. B. et al. Immature dendritic cells generated with low doses of GM-CSF in the absence of IL-4 are maturation resistant and prolong allograft survival in vivo. Eur. J. Immunol. 30, 1813-1822 (2000)
33. Lechler, R., Ng, W. F. & Steinman, R. M. Dendritic cells in transplantation-friend or foe? Immunity 14, 357-368 (2001).
34. Nishimura, H., Nose, M., Hiai, H., Minato, N. & Honjo, T. Development of lupus-like autoimmune diseases by disruption of the PD-1 gene encoding an ITIM motif-carrying immunoreceptor. Immunity 11, 141-151 (1999).
35. Shultz, L. D. et al. Mutations at the murine motheaten locus are within the hematopoietic cell protein-tyrosine phosphatase (Hcph) gene. Cell 73, 1445-1454 (1993).
36. Steinman, R. M. The dendritic cell system and its role in immunogenicity. Annu. Rev. Immunol. 9, 271-296 (1991).
37. Steinman, R. M., Turley, S., Mellman, I. & Inaba, K. The induction of tolerance by dendritic cells that have captured apoptotic cells. J. Exp. Med. 191, 411-416 (2000).
38. Jonuleit, H., Schmitt, E., Schuler, G., Knop, J. & Enk, A. H. Induction of interleukin 10-producing, nonproliferating CD4(+) T cells with regulatory properties by repetitive stimulation with allogeneic immature human dendritic cells. J. Exp. Med. 192, 1213-1222 (2000).
39. Moore, K. W., de Waal Malefyt, R., Coffman, R. L. & O'Garra, A. Interleukin-10 and the interleukin-10 receptor. Annu. Rev. Immunol. 19, 683-765 (2001).
40. Suciu-Foca Cortesini, N. et al. Distinct mRNA microarray profiles of tolerogenic dendritic cells. Hum. Immunol. 62, 1065-1072 (2001).
41. Lanzavecchia, A. Immunology. Licence to kill. Nature 393, 413-414 (1998).
42. Banchereau, J. et al. Immunbiology of dendritic cells. Annu. Rev. Immunol. 18, 767-811 (2000).
43. Ciubotariu, R. et al. Persistent allopeptide reactivity and epitope spreading in chronic rejection of organ allografts. J. Clin. Invest. 101, 398-405 (1998).
44. Bender, A., Sapp, M., Schuler, G., Steinman, R. M. & Bhardwaj, N. Improved methods for the generation of dendritic cells from nonproliferating progenitors in human blood. J. Immunol. Meth. 196, 121-135 (1996).
45. Romani, N., et al. Generation of mature dendritic cells from human blood. An improved method with special regard to clinical applicability. J. Immunol. Meth. 196, 137-151 (1996).
46. Wong, H., Anderson, W. D., Cheng, T. & Riabowol, K. T. Monitoring mRNA expression by polymerase chain reaction: The “primer-dropping” method. Analytical Biochem. 223, 251-258 (1994).
47. Cherry, S. R., Biniszkiewicz, D., van Parijs, L., Baltimore, D. & Jaenisch, R. Retroviral expression in Embryonic stem cells and hematopoetic stem cells. Mol. Cell. Biol. 20, 7419-7426 (2000).
48. Bahnson, A. B. et al. Centrifugal enhancement of retroviral mediated gene transfer. J. Virol. Meth. 54, 131-143 (1995).
49. Chang, C-C., Zhang, J., Lombardi, L., Neri, A. & Dalla-Favera, R. Rearranged NF-κB-2 genes in lymphoid neoplasms code for constitutively active nuclear transactivators. Mol. Cell. Biol. 15, 5180-5187.

### References for the Second Series of Experiments

1. Sakaguchi, S. Regulatory T cells: key controllers of immunologic self-tolerance. Cell 101:455-458 (2000).
2. Roncarolo, M-G., Levings, M. K., & Traversari, C. Differentiation of T regulatory cells by immature dendritic cells. J. Exp. Med. 193:F5-F9 (2001).
3. Shevach E. M. Certified professionals: CD4(+)CD25(+) suppressor T cells. J Exp Med. 193:F41-6 (2001).
4. Shevach, E. M. Regulatory T cells in autoimmunity. Annu. Rev. Immunol. 18:423-449 (2000).
5. Levings M K, Sangregorio R, Roncarolo M G. Human CD25(+)CD4(+) T regulatory cells suppress naive and memory T cell proliferation and can be expanded in vitro without loss of function. J Exp Med. 193:1295-302 (2001).
6. Takahashi, T. et al. Immunologic self-tolerance maintained by CD25⁺ CD4⁺ regulatory T cells constitutively expressing cytotoxic T lymphocyte-associated antigen 4. J. Exp. Med. 192:303-310 (2000).
7. Groux, H. et al. A CD4⁺ T-cell subset inhibits antigen-specific T-cell responses and prevents colitis. Nature 389:737-742 (1997).
8. Filaci G. et al. Impairment of CD8(+) T suppressor cell function in patients with active systemic lupus erythematosus. J. Immunol. 166:6452-7 (2001).
9. Dhodapkar, M. V., Steinman, R. M., Krasovsky, J., Munz, C., & Bhardwaj, N. Antigen-specific inhibition of effector T cell function in humans after injection of immature dendritic cells. J. Exp. Med. 193:233-238 (2001).
10. Liu, Z., Tugulea, S., Cortesini, R., & Suciu-Foca, N. Specific suppression of T helper alloreactivity by allo-MHC class I-restricted CD8⁺ CD28⁻ T cells. Int. Immunol. 10:775-783 (1998).
11. Ciubotariu, R. et al. Specific suppression of human CD4⁺ Th cell responses to pig MHC antigens by CD8⁺ CD28⁻ regulatory T cells. J. Immunol. 161:5193-5202 (1998).
12. Jiang, S., et al. Induction of MHC-class I restricted human suppressor T cells by peptide priming in vitro. Human Immunol. 59:690-699 (1998).
13. Colovai, A. I. et al. Induction of xenoreactive CD4⁺ T-cell anergy by suppressor CD8⁺ CD28⁻ T cells. Transplantation 69:1304-1310 (2000).
14. Liu, Z., Tugulea, S., Cortesini, R., Lederman, S., & Suciu-Foca, N. Inhibition of CD40 signaling pathway in antigen presenting cells by T suppressor cells. Human Immunol. 60:568-574 (1999).
15. Li, J. et al. T suppressor lymphocytes inhibit NF-κB-mediated transcription of CD86 gene in APC. J. Immunol. 163:6386-6392 (1999).
16. St. Louis, D. C. et al. Evidence for distinct intracellular signaling pathways in CD34⁺ progenitor to dendritic cell differentiation from a human cell line model. J. Immunol. 162:3237-3248 (1999).
17. Colonna, M., Nakajima, H., & Cella, M. A family of inhibitory and activating Ig-like receptors that modulate function of lymphoid and myeloid cells. Semin. in Immunol. 12:121-127 (2000).
18. Colonna, M. et al. Cutting Edge: Human myelomonocytic cells express an inhibitory receptor for classical and nonclassical MHC class I molecules. J. Immunol. 160:3096-3100 (1998).
19. Ravetch, J. V., & Lanier, L. Immune inhibitory receptors. Science 290:84-88 (2000).
20. Cella, M. et al. A novel inhibitory receptor (ILT3) expressed on monocytes, macrophages, and dendritic cells involved in antigen processing. J. Exp. Med. 185:1743-1751 (1997).
21. Dalod, M, et al. Altered ex vivo balance between CD28+ and CD28− cells within HIV-specific CD8+ T cells of HIV-seropositive patients. Eur J. Immunol. 1:38-44 (1999).
22. Appay, V, et al. HIV-specific CD8(+) T cells produce antiviral cytokines but are impaired in cytolytic function. J Exp Med. 192:63-75 (2000).
23. Borthwick N J, et al. Lymphocyte activation in HIV-1 infection. II. Functional defects of CD28− T cells. AIDS. 4:431-41 (1994).
24. De Maria, A., and Moretta L. HLA-class I-specific inhibitory receptors in HIV-1 infection. Hum Immunol. 61:74-81 (2000).
25. De Maria A, et al. Multiple HLA-class I-specific inhibitory NK receptor expression and IL-4/IL-5 production by CD8+ T-cell clones in HIV-1 infection. Immunol Lett. 72:179-82 (2000).
26. Andre P, et al. Differential regulation of killer cell Ig-like receptors and CD94 lectin-like dimers on NK and T lymphocytes from HIV-1-infected individuals. Eur J. Immunol. 4:1076-85 (1999).
27. Colonna M, Nakajima H, Navarro F, Lopez-Botet M. A novel family of Ig-like receptors for HLA class I molecules that modulate function of lymphoid and myeloid cells. J Leukoc Biol. 66:375-81 (1999).
28. Steinman, R. M. The dendritic cell system and its role in immunogenicity. Annu. Rev. Immunology. 9:271-296 (1991).
29. Steinman R M, Turley S, Mellman I, Inaba K. The induction of tolerance by dendritic cells that have captured apoptotic cells. J. Exp. Med. 191:411-416 (2000).
30. Jonuleit H, Schmitt E, Schuler G, Knop J, Enk A H. Induction of interleukin 10-producing, nonproliferating CD4(+) T cells with regulatory properties by repetitive stimulation with allogeneic immature human dendritic cells. J. Exp. Med. 192:1213-22 (2000).
31. Moore K. W., de Waal Malefyt, R., Coffman, R. L., and A. O'Garra. Interleukin-10 and the interleukin-10 receptor. Annu Rev Immunol. 19:683-765 (2001).
32. Rea D., et al. Glucocorticoids transform CD40-triggering of dendritic cells into an alternative activation pathway resulting in antigen-presenting cells that secrete IL-10. Blood. 95:3162-7 (2000).
33. Penna G, Adorini L. 1 Alpha,25-dihydroxyvitamin D3 inhibits differentiation, maturation, activation, and survival of dendritic cells leading to impaired alloreactive T cell activation. J. Immunol. 164:2405-11 (2000).
34. Lutz, M. B., et al. Immature dendritic cells generated with low doses of GM-CSF in the absence of IL-4 are maturation resistant and prolong allograft survival in vivo. Eur J. Immunol. 30:1813-22 (2000).
35. Lechler R., Ng, W. F., and R. M. Steinman. Dendritic cells in transplantation—friend or foe? Immunity. 14:357-68 (2001).
36. Mingari, M. C., Moretta, A., Moretta L. Regulation of KIR expression in human T cells: a safety mechanism that may impair protective T-cell responses. Immunol. Today 19:153-157 (1998).
37. Mingari, M. C., et al. Human CD8+ T lymphocyte subsets that express HLA class I-specific inhibitory receptors represent oligoclonally or monoclonally expanded cell populations. Proc. Natl. Acad. Sci. U.S.A. 93:12433-8 (1996).
38. Ciubotariu, R., et al. Detection of T suppressor cells in patients with organ allografts. Hum. Immunol. 62:15-20 (2001).
39. Nishimura, H., Nose, M., Hiai, H., Minato, N., Honjo, T. Development of lupus-like autoimmune diseases by disruption of the PD-1 gene encoding an ITIM motif-carrying immunoreceptor. Immunity 11:141-151 (1999).
40. Shultz, L. D. et al. Mutations at the murine motheaten locus are within the hematopoietic cell protein-tyrosine phosphatase (Hcph) gene. Cell 73:1445-1454 (1993).
41. Banchereau, J. et al. Immunbiology of dendritic cells. Annu. Rev. Immunol. 18:767-811 (2000).
42. Ciubotariu, R. et al. Persistent allopeptide reactivity and epitope spreading in chronic rejection of organ allografts. J. Clin. Invest. 101:398-405 (1998).
43. Dixon, W. J., Brown, M. B., Engelman, L., ans R. I. Jannrich. BMDP statistical sortware. University of California Press, 1990.
44. Wong, H., Anderson, W. D., Cheng, T., & Riabowol, K. T. Monitoring mRNA expression by polymerase chain reaction: The “primer-dropping” method. Analytical. Biochem. 223, 251-258 (1994).
45. Cherry, S. R., Biniszkiewicz, D., van Parijs, L., Baltimore, D., and R. Jaenisch. Retroviral expression in Embryonic stem cells and hematopietic stem cells. Mol. Cell. Biol. 20: 7419-7426 (2000).
46. Chang, C.-C., Zhang, J., Lombardi, L., Neri, A., and R. Dalla-Favera. Rearranged NFKB-2 genes in lymphoid neoplasms code for constitutively active nuclear transactivators. Mol. Cell. Biol. 15:5180-5187.

1-27. (canceled)

28. A method for inhibiting anergizing function in an antigen presenting cell, the method comprising the step of contacting the antigen presenting cell with an antibody that specifically binds to ILT3 and/or ILT4.

29. The method of claim 28, wherein the antigen-presenting cell is a human antigen-presenting cell.

30. The method of claim 28, wherein the contacting step is performed in vivo:

31-32. (canceled)

33. The method of claim 28, wherein the antigen-presenting cell is a dendritic cell or a monocyte.

34. A method for inhibiting T cell tolerization in a patient with AIDS, which method comprises the step of administering to the subject a composition comprising an effective amount of an antibody that specifically binds to ILT3 and/or ILT4 and a pharmaceutically acceptable carrier.

35. A method for treating AIDS in an afflicted subject, which method comprises the step of administering to the subject a composition comprising a therapeutically effective amount of an antibody that specifically binds to ILT3 and/or ILT4 and a pharmaceutically acceptable carrier.

36. The method of claim 34, wherein the subject is human.

37. The method of claim 35, wherein the subject is human.

38-66. (canceled)
